(19) United States
(12) Patent Application Publication
(10) Pub. No.: US 2019/0031615 A1
(43) Pub. Date: Jan. 31, 2019

(54) 3-ETHYL-3-PHENYLAZEPANE DERIVATIVES HAVING MULTIMODAL ACTIVITY AGAINST PAIN

(71) Applicant: LABORATORIOS DEL DR. ESTEVE S.A., Barcelona (ES)

(72) Inventors: Ramón MERCE-VIDAL, Barcelona (ES); Carmen ALMANSA-ROSALES, Barcelona (ES); Monica GARCIA-LOPEZ, Barcelona (ES)

(21) Appl. No.: 16/069,529

(22) PCT Filed: Jan. 13, 2017
(86) PCT No.: PCT/EP2017/000039

§ 371 Date: Jul. 12, 2018

(30) Foreign Application Priority Data
Jan. 15, 2016 (EP) 16382016.0

(51) Int. Cl. C07D 223/04; C07D 401/12

(52) U.S. Cl. CPC C07D 223/04; C07D 401/12

(57) ABSTRACT

The present invention relates to 3-ethyl-3-phenylazepane derivatives having dual pharmacological activity towards both the sigma (σ) receptor and the μ-opioid receptor, to processes of preparation of such compounds, to pharmaceutical compositions comprising them, and to their use in therapy, in particular for the treatment of pain.

### FIELD OF THE INVENTION

The present invention relates to compounds having dual pharmacological activity towards both the sigma (a) receptor, and the μ-opioid receptor (MOR or mu-opioid receptor) and more particularly to 3-ethyl-3-phenylazepane derivatives having this pharmacological activity, to processes of preparation of such compounds, to pharmaceutical compositions comprising them, and to their use in therapy, in particular for the treatment of pain.

### BACKGROUND OF THE INVENTION

The adequate management of pain constitutes an important challenge, since currently available treatments provide in many cases only modest improvements, leaving many patients unrelieved [Turk D C, Wilson H D, Cahana A. Treatment of chronic non-cancer pain. Lancet 377, 2226-2235 (2011)]. Pain affects a big portion of the population with an estimated prevalence of around 20% and its incidence, particularly in the case of chronic pain, is increasing due to the population ageing. Additionally, pain is clearly related to comorbidities, such as depression, anxiety and insomnia, which lead to important productivity losses and socio-economic burden [Goldberg D S, McGee S J. Pain as a global public health priority. BMC Public Health. 11, 770 (2011)]. Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

As mentioned before, there are few available therapeutic classes for the treatment of pain, and opioids are among the most effective, especially when addressing severe pain states. They act through three different types of opioid receptors (mu, kappa and gamma) which are transmembrane G-protein coupled receptors (GPCRs). Still, the main analgesic action is attributed to the activation of the μ-opioid receptor (MOR). However, the general administration of MOR agonists is limited due to their important side effects, such as constipation, respiratory depression, tolerance, emesis and physical dependence [Meldrum, M. L. (Ed.). Opioids and Pain Relief: A Historical Perspective. Progress in Pain Research and Management, Vol 25. IASP Press, Seattle, 2003]. Additionally, MOR agonists are not optimal for the treatment of chronic pain as indicated by the diminished effectiveness of morphine against chronic pain conditions. This is especially proven for the chronic pain conditions of neuropathic or inflammatory origin, in comparison to its high potency against acute pain. The finding that chronic pain can lead to MOR down-regulation may offer a molecular basis for the relative lack of efficacy of morphine in long-term treatment settings [Dickenson, A. H., Suzuki, R. Opioids in neuropathic pain: Clues from animal studies. Eur J Pain 9, 113-6 (2005)]. Moreover, prolonged treatment with morphine may result in tolerance to its analgesic effects, most likely due to treatment-induced MOR down-regulation, internalization and other regulatory mechanisms. As a consequence, long-term treatment can result in substantial increases in dosing in order to maintain a clinically satisfactory pain relief, but the narrow therapeutic window of MOR agonists finally results in unacceptable side effects and poor patient compliance.

The sigma-1 (σ₁) receptor was discovered 35 years ago and initially assigned to a new subtype of the opioid family, but later on and based on the studies of the enantiomers of SKF-10,047, its independent nature was established. The first link of the σ₁ receptor to analgesia was established by Chien and Pastemak [Chien C C, Pastemak G W. Sigma antagonists potentiate opioid analgesia in rats. Neurosci. Lett. 190, 137-9 (1995)], who described it as an endogenous anti-opioid system, based on the finding that σ₁ receptor agonists counteracted opioid receptor mediated analgesia, while σ₁ receptor antagonists, such as haloperidol, potentiated it.

Many additional preclinical evidences have indicated a clear role of the σ₁ receptor in the treatment of pain [Zamanillo D, Romero L, Merlos M, Vela J M, Sigma 1 receptor: A new therapeutic target for pain. Eur. J. Pharmacol, 716, 78-93 (2013)]. The development of the σ₁ receptor knockout mice, which show no obvious phenotype and perceive normally sensory stimuli, was a key milestone in this endeavour. In physiological conditions the responses of the σ₁ receptor knockout mice to mechanical and thermal stimuli were found to be undistinguishable from WT ones but they were shown to possess a much higher resistance to develop pain behaviours than WT mice when hypersensitivity entered into play. Hence, in the σ₁ receptor knockout mice capsaicin did not induce mechanical hypersensitivity, both phases of formalin-induced pain were reduced, and cold and mechanical hypersensitivity were strongly attenuated after partial sciatic nerve ligation or after treatment with paclitaxel, which are models of neuropathic pain. Many of these actions were confirmed by the use of σ₁ receptor antagonists and led to the advancement of one compound, SIRA, into clinical trials for the treatment of different pain states. Compound SIRA exerted a substantial reduction of neuropathic pain and anhedonic state following nerve injury (i.e., neuropathic pain conditions) and, as demonstrated in an operant self-administration model, the nerve-injured mice, but not sham-operated mice, acquired the operant responding to obtain it (presumably to get pain relief), indicating that σ₁ receptor antagonism relieves neuropathic pain and also address some of the comorbidities (i.e., anhedonia, a core symptom in depression) related to pain states.

Pain is multimodal in nature, since in nearly all pain states several mediators, signaling pathways and molecular mechanisms are implicated. Consequently, monomodal therapies fail to provide complete pain relief. Currently, combining existing therapies is a common clinical practice and many efforts are directed to assess the best combination of available drugs in clinical studies [Mao J, Gold M S, Backonja M. Combination drug therapy for chronic pain: a call for more clinical studies. J. Pain 12, 157-166 (2011)]. Hence, there is an urgent need for innovative therapeutics to address this unmet medical need.

As mentioned previously, opioids are among the most potent analgesics but they are also responsible for various adverse effects which seriously limit their use.

Accordingly, there is still a need to find compounds that have an alternative or improved pharmacological activity in the treatment of pain, being both effective and showing the desired selectivity, and having good “drugability” properties, i.e. good pharmaceutical properties related to administration, distribution, metabolism and excretion.

Thus, the technical problem can therefore be formulated as finding compounds that have an alternative or improved pharmacological activity in the treatment of pain.

In view of the existing results of the currently available therapies and clinical practices, the present invention offers a solution by combining in a single compound binding to two different receptors relevant for the treatment of pain. This was mainly achieved by providing the compounds according to the invention that bind both to the μ-opioid receptor and to the σ₁ receptor.

### SUMMARY OF THE INVENTION

In this invention a family of structurally distinct 3-ethyl-3-phenylazepane derivatives which have a dual pharmacological activity towards both the sigma (σ) receptor, and the μ-opioid receptor was identified thus solving the above problem of identifying alternative or improved pain treatments by offering such dual compounds.

The invention is in one aspect directed to a compound having a dual activity binding to the σ₁ receptor and the μ-opioid receptor for use in the treatment of pain.

As this invention is aimed at providing a compound or a chemically related series of compounds which act as dual ligands of the σ₁ receptor and the μ-opioid receptor it is a very preferred embodiment if the compound has a binding expressed as Kᵢ which is preferably <1000 nM for both receptors, more preferably <500 nM, even more preferably <100 nM.

The invention is directed in a main aspect to a compound of general Formula (I),

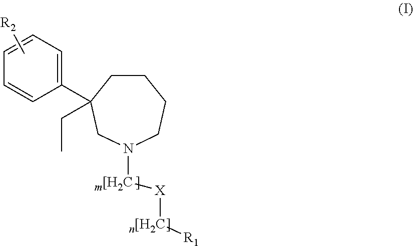

wherein R₁, R₂, m, n and X are as defined below in the detailed description.

A further object of the invention refers to the processes for preparation of compounds of general formula (I).

A still further object of the invention refers to the use of intermediate compounds for the preparation of a compound of general formula (I).

It is also an object of the invention a pharmaceutical composition comprising a compound of formula (I).

Finally, it is an object of the invention the use of compound as a medicament and more particularly for the treatment of pain and pain related conditions.

### DETAILED DESCRIPTION OF THE INVENTION

The invention is directed to a family of structurally distinct 3-ethyl-3-phenylazepane derivatives which have a dual pharmacological activity towards both the sigma (σ) receptor and the μ-opioid receptor, thus solving the above problem of identifying alternative or improved pain treatments by offering such dual compounds.

The invention is in one aspect directed to a compound having a dual activity binding to the σ₁ receptor and the μ-opioid receptor for use in the treatment of pain.

As this invention is aimed at providing a compound or a chemically related series of compounds which act as dual ligands of the σ₁ receptor and the μ-opioid receptor it is a preferred embodiment if the compound has a binding expressed as K which is preferably <1000 nM for both receptors, more preferably <500 nM, even more preferably <100 nM.

The applicant has surprisingly found that the problem on which the present invention is based can be solved by using a multimodal balanced analgesic approach combining two different synergistic activities in a single drug (i.e., dual ligands which are bifunctional and bind to μ-opioid receptor and to σ₁ receptor), thereby enhancing the opioid analgesia through the σ₁ activation without increasing the undesirable side effects. This supports the therapeutic value of a dual MOR/σ₁ receptor compound whereby the σ₁ receptor binding component acts as an intrinsic adjuvant of the MOR binding component.

This solution offered the advantage that the two mechanisms complement each other in order to treat pain and chronic pain using lower and better tolerated doses needed based on the potentiation of analgesia but avoiding the adverse events of μ-opioid receptor agonists.

A dual compound that possess binding to both the μ-opioid receptor and to the σ₁ receptor shows a highly valuable therapeutic potential by achieving an outstanding analgesia (enhanced in respect to the potency of the opioid component alone) with a reduced side-effect profile (safety margin increased compared to that of the opioid component alone) versus existing opioid therapies.

Advantageously, the dual compounds according to the present invention would in addition show one or more the following functionalities: σ₁ receptor antagonism and μ-opioid receptor agonism. It has to be noted, though, that both functionalities “antagonism” and “agonism” are also sub-divided in their effect into subfunctionalities like partial agonism or inverse agonism.

Accordingly, the functionalities of the dual compound should be considered within a relatively broad bandwidth.

An antagonist on one of the named receptors blocks or dampens agonist-mediated responses. Known subfunctionalities are neutral antagonists or inverse agonists.

An agonist on one of the named receptors increases the activity of the receptor above its basal level. Known subfunctionalities are full agonists, or partial agonists.

In addition, the two mechanisms complement each other since MOR agonists are only marginally effective in the treatment of neuropathic pain, while σ₁ receptor antagonists show outstanding effects in preclinical neuropathic pain models. Thus, the or receptor component adds unique analgesic actions in opioid-resistant pain. Finally, the dual approach has clear advantages over MOR agonists in the treatment of chronic pain as lower and better tolerated doses would be needed based on the potentiation of analgesia but not of the adverse events of MOR agonists.

A further advantage of using designed multiple ligands is a lower risk of drug-drug interactions compared to cocktails or multi-component drugs, thus involving simpler pharmacokinetics and less variability among patients. Additionally, this approach may improve patient compliance and broaden the therapeutic application in relation to monomechanistic drugs, by addressing more complex aetiologies. It is also seen as a way of improving the R&D output obtained using the “one drug-one target” approach, which has been questioned over the last years [Bomot A, Bauer U, Brown A, Firth M, Hellawell C, Engkvist O. Systematic Exploration of Dual-Acting Modulators from a Combined Medicinal Chemistry and Biology Perspective. J. Med. Chem, 56, 1197-1210 (2013)].

In a particular aspect, the present invention is directed to compounds of general Formula (I):

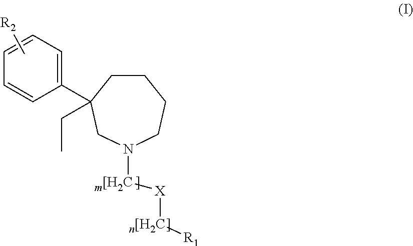

wherein
m is 1, 2 or 3;
n is 0, 1 or 2;
X is selected from a —CH₂N(R_{1′})—, —C(O)N(R_{1′})— and —CH₂O—;
R₁ is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl;
R_{1′} is selected from substituted or unsubstituted C₁₋₆ alkyl, substituted or unsubstituted C₂₋₆ alkenyl, substituted or unsubstituted C₂₋₆ alkynyl;
alternatively, when X is —CH₂N(R_{1′})— or —C(O)N(R_{1′})—, R₁ and R_{1′} taken together with the connecting N—[CH₂]ₙ atoms may form a substituted or unsubstituted up to 6-member heterocyclyl;
R₂ is selected from hydrogen, halogen, —R₄, —OR₄, —NO₂, —NR₄R_{4′″}, —NR₄C(O)R_{4′}, —NR₄S(O)₂R_{4′}, —S(O)₂NR₄R_{4′}, —NR₄C(O)NR_{4′}R_{4″}, —SR₄, —S(O)R₄, —S(O)₂R₄, —OS(O)₂R₄, —CN, haloalkyl, haloalkoxy, —C(O)OR₄, —C(O)NR₄R_{4′}, —OCH₂CH₂OH, —NR₄S(O)₂NR_{4′}R_{4″} and —C(CH₃)₂OR₄; wherein
R₄, R_{4′} and R_{4″} are independently selected from hydrogen, unsubstituted C₁₋₆ alkyl, unsubstituted C₂₋₆ alkenyl, and unsubstituted C₂₋₆ alkynyl;
R_{4′″} is selected from hydrogen, unsubstituted C₁₋₆ alkyl, unsubstituted C₂₋₆ alkenyl, unsubstituted C₂₋₆ alkynyl and -Boc.

In a particular aspect, the present invention is—in a preferred embodiment—directed to compounds of general Formula (I):

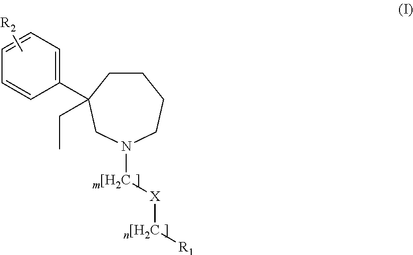

wherein
m is 1, 2 or 3;
n is 0, 1 or 2;
X is selected from a —CH₂N(R_{1′})—, —C(O)N(R_{1′})— and —CH₂O—;
R₁ is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl;
R_{1′} is selected from substituted or unsubstituted C₁₋₆ alkyl, substituted or unsubstituted C₂₋₆ alkenyl, substituted or unsubstituted C₂₋₆ alkynyl;
alternatively, when X is —CH₂N(R_{1′})— or —C(O)N(R_{1′})—, R₁ and R_{1′} taken together with the connecting N—[CH₂]ₙ atoms may form a substituted or unsubstituted up to 6-member heterocyclyl;
wherein the alkyl, alkenyl or alkynyl in R₁ or R_{1′}, if substituted, is substituted with one or more substituents selected from —OR₃, halogen, —CN, haloalkyl, haloalkoxy and —NR₃R_{3′″};
wherein
R₃ is selected from hydrogen, unsubstituted C₁₋₆ alkyl, unsubstituted C₂₋₆ alkenyl and unsubstituted C₂₋₆ alkynyl;
and wherein R_{3′″} is selected from hydrogen, unsubstituted C₁₋₆ alkyl, unsubstituted C₂₋₆ alkenyl, unsubstituted C₂₋₆ alkynyl and -Boc;
   R₂ is selected from hydrogen, halogen, —R₄, —OR₄, —NO₂, —NR₄R_{4′″}, —NR₄C(O)R_{4′}, —NR₄S(O)₂R_{4′}, —S(O)₂NR₄R_{4′}, —NR₄C(O)NR_{4′}N_{4″}, —SR₄, —S(O)R₄, —S(O)₂R₄, —OS(O)₂R₄, —CN, haloalkyl, haloalkoxy, —C(O)OR₄, —C(O)NR₄R_{4′}, —OCH₂CH₂OH, —NR₄S(O)₂NR_{4′}R_{4″} and —C(CH₃)₂OR₄; wherein
R₄, R_{4′}, and R_{4″} are independently selected from hydrogen, unsubstituted C₁₋₆ alkyl, unsubstituted C₂₋₆ alkenyl, and unsubstituted C₂₋₆ alkynyl;
R_{4′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

For clarity sake, in the above Formula (I)
X is selected from a —CH₂N(R_{1′})—, —C(O)N(R_{1′})— and —CH₂O—;
if “X” is “—CH₂N(R_{1′})—”, this means that this results in . . . —(CH₂)ₘ—CH₂N(R_{1′})—(CH₂)ₙ—R₁;
if “X” is “—C(O)N(R_{1′})—”, this means that this results in . . . —(CH₂)ₘ—C(O)N(R_{1′})—(CH₂)ₙ—R₁; and
if “X” is “—CH₂O—”, this means that this results in . . . —(CH₂)ₘ—CH₂O—(CH₂)ₙ—R₁;

These compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof.

In a further embodiment the compound according to the invention of general Formula (I) is a compound of general Formula (I′)

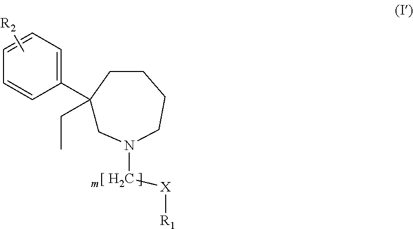

wherein, R₁, R₂, X and m are as defined in the description.

In a further embodiment the compound according to the invention of general Formula (I) is a compound of general Formula (I²′)

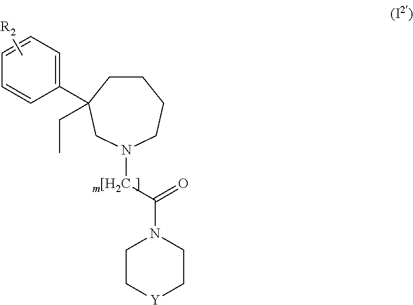

with Y being selected from —O—, —N(R_{1″})— or —CH(R_{1″})—, and R₂ and m are as defined in the description, and wherein
R_{1″} is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl; preferably being selected from hydrogen, unsubstituted C₁₋₄ alkyl, or unsubstituted aryl.

In a further embodiment the compound according to the invention of general Formula (I) is a compound of general Formula (I³′)

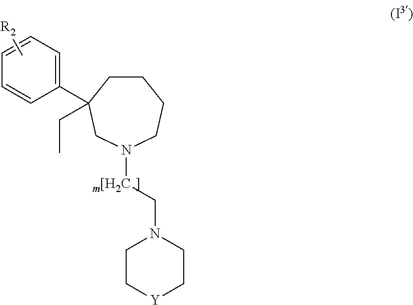

with Y being selected from —O—, —N(R_{1″})— or —CH(R_{1′})—, and R₂ and m are as defined in the description, and wherein
R_{1″} is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl; preferably being selected from hydrogen, unsubstituted C₁₋₄ alkyl, or unsubstituted aryl.

In a further embodiment the compound according to the invention of general Formula (I) is a compound wherein when X is —CH₂N(R_{1′})— or —C(O)N(R_{1′})— and when R₁ and R_{1′} taken together with the connecting N—[CH₂]ₙ atoms or N atom form a substituted or unsubstituted 6-member heterocyclyl, —X—[CH₂]ₙ—R₁ or —X—R₁ is represented by

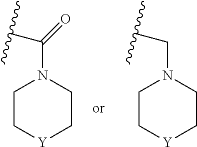

respectively,
with Y being selected from —O—, —N(R_{1″})— or —CH(R_{1″})— and
R_{1″} being selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl; preferably being selected from hydrogen, unsubstituted C₁₋₄ alkyl, or unsubstituted aryl.

This would lead to the following compounds according to Formula (I) (or Formula (I′)) being of general Formulas (I^{2′}) and (I^{3′})

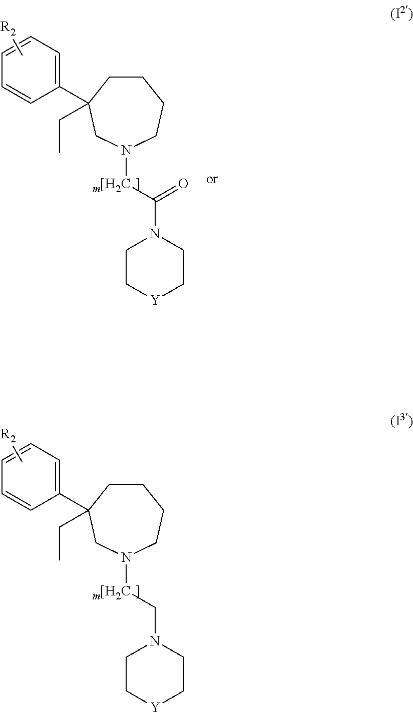

with Y being selected from —O—, —N(R_{1″})— or —CH(R_{1″})— and
R_{1″} being selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl; preferably being selected from hydrogen, unsubstituted C₁₋₄ alkyl, or unsubstituted aryl.

In a further embodiment the compound according to the invention of general Formula (I) is a compound of general Formula (I⁴′)

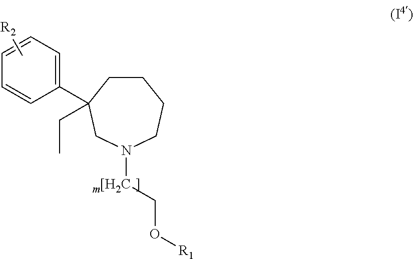

wherein, R₁, R₂ and m are as defined in the description.

In a further preferred embodiment the compound according to the invention of general Formula (I) is a compound of general Formula (I), general Formula (I′), general Formula (I^{2′}), general Formula (I^{3′}) or general Formula (I^{4′}), wherein R² is in meta position on the phenyl-ring.

In a further preferred embodiment the compound according to the invention of general Formula (I) is a compound of general Formula (IA), general Formula (I′A), general Formula (I^{2′}A), general Formula (I^{3′}A) and general Formula (I^{4′}A) shown below, wherein the compound is defined—except for m—as for a compound of general Formula (I), general Formula (I′), general Formula (I^{2′}), general Formula (I^{3′}) and general Formula (I^{4′}) respectively:

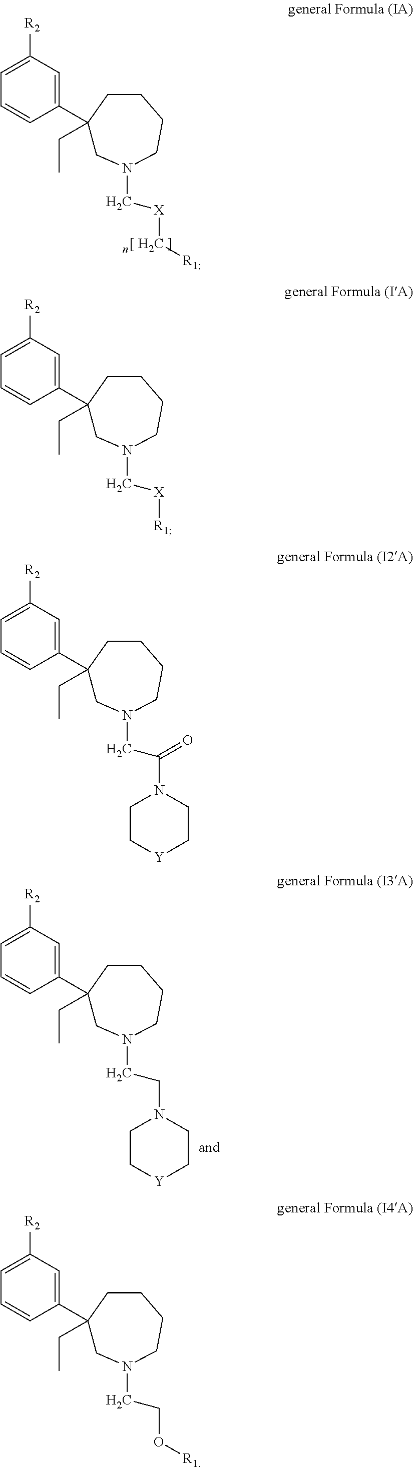

For clarity purposes, all groups and definitions described in the description and referring to compounds of general Formula (I), also apply to compounds of general Formula (I′), (I^{2′}), (I^{3′}) and (I^{4′}), to compounds of general Formula (IA), (I′A), (I²′A), (I³′A) and (I⁴′A) as well as to all the intermediates of synthesis, when those groups are present in the mentioned general Markush formulae, since compounds of general Formula (I′), (I²′), (I³′) and (I⁴′) as well as compounds of general Formula (IA), (I′A), (I²′A), (I³′A) and (I⁴′A) are included in the general Formula (I).

For clarity purposes, the general Markush Formula (I)

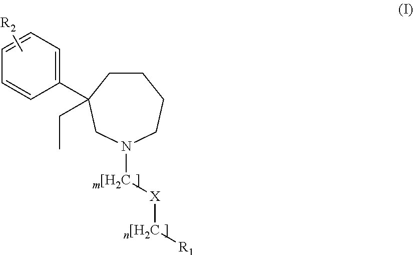

is equivalent to

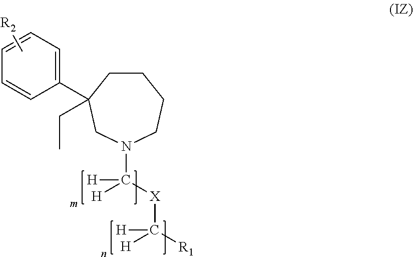

wherein only —[CH₂]— are included into the brackets and m and n mean the number of times that —[CH₂]— is repeated. The same would apply to general Markush Formulae (I′), (I²′), (I³′) and (I⁴′), general Markush Formulae (IA), (I′A), (I²′A), (I³′A) and (I⁴′A) as well as to all the intermediates of synthesis.

In addition, and for clarity purposes, it should further be understood that naturally if m is 0, then X is still present in general Markush Formulae (I′), (I²′), (I³′) and (I⁴′) as well as in all the intermediates of synthesis. It should also further be understood that if n is 0, then X and R₁ are still present in general Markush Formulae (I′), (I²′), (I³′) and (I⁴′), general Markush Formulae (IA), (I′A), (I²′A), (I³′A) and (I⁴′A) as well as in all the intermediates of synthesis.

In the context of this invention, alkyl is understood as meaning saturated, linear or branched hydrocarbons, which may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc. Preferably alkyl is understood in the context of this invention as C₁₋₈alkyl like methyl, ethyl, propyl, butyl, pentyl, hexyl, heptyl, or octyl; preferably is C₁₋₆alkyl like methyl, ethyl, propyl, butyl, pentyl, or hexyl; more preferably is C₁₋₄alkyl like methyl, ethyl, propyl or butyl.

Alkenyl is understood as meaning unsaturated, linear or branched hydrocarbons, which may be unsubstituted or substituted once or several times. It encompasses groups like e.g. —CH═CH—CH₃. The alkenyl radicals are preferably vinyl (ethenyl), allyl (2-propenyl). Preferably in the context of this invention alkenyl is C₂₋₁₀-alkenyl or C₂₋₈-alkenyl like ethylene, propylene, butylene, pentylene, hexylene, heptylene or octylene; or is C₂₋₆-alkenyl like ethylene, propylene, butylene, pentylene, or hexylene; or is C₂₋₆-alkenyl, like ethylene, propylene, or butylenes.

Alkynyl is understood as meaning unsaturated, linear or branched hydrocarbons, which may be unsubstituted or substituted once or several times. It encompasses groups like e.g. —C≡C—CH₃ (1-propinyl). Preferably alkynyl in the context of this invention is C₂₋₁₀-alkynyl or C₂₋₈-alkynyl like ethyne, propyne, butyene, pentyne, hexyne, heptyne, or octyne; or is C₂₋₆-alkynyl like ethyne, propyne, butyene, pentyne, or hexyne; or is C₂₋₄-alkynyl like ethyne, propyne, butyene, pentyne, or hexyne.

In connection with alkyl (also in alkylaryl, alkylheterocyclyl or alkylcycloalkyl), alkenyl, alkynyl and O-alkyl—unless defined otherwise—the term substituted in the context of this invention is understood as meaning replacement of at least one hydrogen radical on a carbon atom by halogen (F, Cl, Br, I), —NR_{c}R_{c′″}, —SR_{c}, —S(O)R_{c}, —S(O)₂R_{c}, —OR_{c}, —C(O)OR_{c}, —CN, —C(O)NR_{c}, haloalkyl, haloalkoxy or —OC₁₋₆alkyl being R_{c} represented by R₃, R₅ (being R_{c′} represented by R_{3′}, R_{5′}; being R_{c″} represented by R_{3″}, R_{5″}, being R_{c′″} represented by R_{3′″}, R_{5′″}); being R_{c″″} represented by R_{c″″}, R_{5″″}) wherein R₁ to R_{6″″} are as defined in the description, and wherein when different radicals R₁ to R_{6″″} are present simultaneously in Formula I they may be identical or different.

Most preferably in connection with alkyl (also in alkylaryl, alkytheterocyclyl or alkylcycloalkyl), alkenyl, alkynyl or O-alkyl, substituted is understood in the context of this invention that any alkyl (also in alkylaryl, alkylheterocyclyl or alkylcycloalkyl), alkenyl, alkynyl or O-alkyl which is substituted is substituted with one or more of halogen (F, Cl, Br, I), —OR_{c}, —CN, —SR_{c}, —S(O)_{c}, and —S(O)₂R_{c}, haloalkyl, haloalkoxy or —OC₁₋₆alkyl being R_{c} represented by R₃, R₅ (being R_{c′} represented by R_{3′}, R_{5′}; being R_{c″} represented by R_{3″}, R_{5″}; being R_{c′″} represented by R_{3′″}, R_{5′″}, being R_{c″″} represented by R_{3″″}, R_{5″″}), wherein R₁ to R_{6″″} are as defined in the description, and wherein when different radicals R₁ to R_{6″″} are present simultaneously in Formula I, they may be identical or different.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH═CHCl₂.

In the context of this invention haloalkyl is understood as meaning an alkyl being substituted once or several times by a halogen (selected from F, Cl, Br, I). It encompasses e.g. —CH₂Cl, —CH₂F, —CHCl₂, —CHF₂, —CHCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂Cl, —CH₂F, —CHCl₂, —CHF₂, and —CF₃.

In the context of this invention haloalkoxy is understood as meaning an —O-alkyl being substituted once or several times by a halogen (selected from F, Cl, Br, I). It encompasses e.g. —OCH₂Cl, —OCH₂F, —OCHCl₂, —OCHF₂, —OCCl₃, —OCF₃ and —OCH₂—CHCl₂. Preferably haloalkyl is understood in the context of this invention as halogen-substituted —OC₁₋₄-alkyl representing halogen substituted C1-, C2-, C3- or C4-alkoxy. The halogen-substituted alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention cycloalkyl is understood as meaning saturated and unsaturated (but not aromatic) cyclic hydrocarbons (without a heteroatom in the ring), which can be unsubstituted or once or several times substituted. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantly. Preferably in the context of this invention cycloalkyl is C₃₋₈cycloalkyl like cyclopropyl, cyclobutyl, cyclopentyl, cyclohexyl, cycloheptyl, or cyclooctyl; or is C₃₋₇cycloalkyl like cyclopropyl, cyclobutyl, cyclopentyl, cyclohexyl, or cycloheptyl; or is C₃₋₆cycloalkyl like cyclopropyl, cyclobutyl, cyclopentyl or cyclohexyl, especially cyclopentyl or cyclohexyl.

Aryl is understood as meaning 5 to 18 membered mono or polycyclic ring systems with at least one aromatic ring but without heteroatoms even in only one of the rings. Examples are phenyl, naphthyl, fluoranthenyl, fluorenyl, tetralinyl or indanyl, 9H-fluorenyl or anthracenyl radicals, which can be unsubstituted or once or several times substituted. Most preferably aryl is understood in the context of this invention as phenyl, naphtyl or anthracenyl, preferably is phenyl.

A heterocyclyl radical or group (also called heterocyclyl hereinafter) is understood as meaning 5 to 18 membered mono or polycyclic heterocyclic ring systems, with at least one saturated or unsaturated ring which contains one or more heteroatoms from the group consisting of nitrogen, oxygen and/or sulfur in the ring. A heterocyclic group can also be substituted once or several times.

Examples include non-aromatic heterocyclyls such as tetrahydropyrane, oxazepane, morpholine, piperidine, pyrrolidine as well as heteroaryls such as furan, benzofuran, thiophene, benzothiophene, pyrrole, pyridine, pyrimidine, pyrazine, quinoline, isoquinoline, phthalazine, thiazole, benzothiazole, indole, benzotriazole, carbazole and quinazoline.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or polycyclic heterocyclic ring system of one or more rings of which at least one aromatic ring contains one or more heteroatoms from the group consisting of nitrogen, oxygen and/or sulfur in the ring; preferably is an aromatic 5 to 18 membered mono or polycyclic heterocyclic ring system of one or two rings of which at least one aromatic ring contains one or more heteroatoms from the group consisting of nitrogen, oxygen and/or sulfur in the ring, more preferably is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, thiophene and benzimidazole;
the non-aromatic heterocyclyl is a 5 to 18 membered mono or polycyclic heterocyclic ring system of one or more rings of which at least one ring—with this (or these) ring(s) then not being aromatic—contains one or more heteroatoms from the group consisting of nitrogen, oxygen and/or sulfur in the ring; preferably is a 5 to 18 membered mono or polycyclic heterocyclic ring system of one or two rings of which one or both rings—with this one or two rings then not being aromatic—contain/s one or more heteroatoms from the group consisting of nitrogen, oxygen and/or sulfur in the ring, more preferably is selected from oxazepam, pyrrolidine, piperidine, piperazine, tetrahydropyran, morpholine, indoline, oxopyrrolidine, benzodioxane, oxetane, especially is benzodioxane, morpholine, tetrahydropyran, piperidine, oxopyrrolidine, oxetane and pyrrolidine.

Preferably in the context of this invention heterocyclyl is defined as a 5 to 18 membered mono or polycyclic heterocyclic ring system of one or more saturated or unsaturated rings of which at least one ring contains one or more heteroatoms from the group consisting of nitrogen, oxygen and/or sulfur in the ring. Preferably it is a 5 to 18 membered mono or polycyclic heterocyclic ring system of one or two saturated or unsaturated rings of which at least one ring contains one or more heteroatoms from the group consisting of nitrogen, oxygen and/or sulfur in the ring.

Preferred examples of heterocyclyls include oxetane, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyrane, pyrazole, imidazole, piperidine, thiophene, indole, benzimidazole, pyrrolo[2,3b]pyridine, benzoxazole, oxopyrrolidine, pyrimidine, oxazepane, oxetane and pyrrolidine.

In the context of this invention oxopyrrolidine is understood as meaning pyrrolidin-2-one.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times. Preferably alkylaryl is understood as meaning an aryl group (see above) being connected to another atom through 1 to 4 (—CH₂—) groups. Most preferably alkylaryl is benzyl (i.e. —CH₂-phenyl).

In the context of this invention alkylheterocyclyl is understood as meaning an heterocyclyl group being connected to another atom through a C₁₋₆-alkyl (see above) which may be branched or linear and is unsubstituted or substituted once or several times. Preferably alkylheterocyclyl is understood as meaning an heterocyclyl group (see above) being connected to another atom through 1 to 4 (—CH₂—) groups. Most preferably alkylheterocyclyl is —CH₂-pyridine.

In the context of this invention alkylcycloalkyl is understood as meaning an cycloalkyl group being connected to another atom through a C₁₋₆-alkyl (see above) which may be branched or linear and is unsubstituted or substituted once or several times. Preferably alkylcycloalkyl is understood as meaning a cycloalkyl group (see above) being connected to another atom through 1 to 4 (—CH₂) groups. Most preferably alkylcycloalkyl is —CH₂-cyclopropyl.

Preferably, the aryl is a monocyclic aryl. More preferably the aryl is a 5, 6 or 7 membered monocyclic aryl. Even more preferably the aryl is a 5 or 6 membered monocyclic aryl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with aryl (including alkyl-aryl), cycloalkyl (including alkyl-cycloalkyl), or heterocyclyl (including alkyl-heterocyclyl), substituted is understood—unless defined otherwise—as meaning substitution of the ring-system of the aryl or alkyl-aryl, cycloalkyl or alkyl-cycloalkyl; heterocycyl or alkyl-heterocyclyl with one or more of halogen (F, Cl, Br, I), —R_{c}, —OR_{c}, —CN, —NO₂, —NR_{c}R_{c′″}, —C(O)OR_{c}, NR_{c}C(O)R_{c′}, —C(O)NR_{c}R_{c′″}, —NR_{c}S(O)₂R_{c′}, ═O, —OCH₂CH₂OH, —NR_{c}C(O)NR_{c′}R_{c″}, —S(O)₂NR_{c}R_{c′}, —NR_{c}S(O)₂NR_{c′}R_{c″}, haloalkyl, haloalkoxy, —SR_{c}, —S(O)R_{c}, —S(O)₂R_{c} or C(CH₃)OR_{c′}; NR_{c}R_{c′″}, with R_{c} and R_{c′″} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C₁₋₆ alkyl (alkoxy); a saturated or unsaturated, linear or branched, substituted or unsubstituted —S—C₁₋₆ alkyl; a saturated or unsaturated, linear or branched, substituted or unsubstituted —C(O)—C₁₋₆ alkyl-group; a saturated or unsaturated, linear or branched, substituted or unsubstituted —C(O)—O—C₁₋₆ alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl, being R_{c} one of R₃ or R₆, (being R_{c′} one of R_{3′} or R_{6′}; being R_{c″} one of R_{3″} or R_{6″}; being R_{c′″} one of R_{3′″} or R_{6′″}; being R_{c″″} one of R_{3″″} or R_{6″″}), wherein R₁ to R_{6″″} are as defined in the description, and wherein when different radicals R₁ to R_{6″″} are present simultaneously in Formula I they may be identical or different.

Most preferably in connection with aryl (including alkyl-aryl), cycloalkyl (including alkyl-cycloalkyl), or heterocyclyl (including alkyl-heterocyclyl), substituted is understood in the context of this invention that any aryl, cycloalkyl and heterocyclyl which is substituted is substituted (also in an alyklaryl, alkylcycloalkyl or alkylheterocyclyl) with one or more of halogen (F, Cl, Br, I), —R_{c}, —OR_{c}, —CN, —NO₂, —NR_{c}R_{c′″}, NR_{c}C(O)R_{c′}, —NR_{c}S(O)₂R_{c′}, ═O, haloalkyl, haloalkoxy, C(CH₃)OR_{c} or —OC₁₋₄ alkyl being unsubstituted or substituted with one or more of OR_{c} or halogen (F, Cl, I, Br), —CN, or —C₁₋₄alkyl being unsubstituted or substituted with one or more of OR_{c} or halogen (F, Cl, I, Br), being R_{c} one of R₃ or R₆, (being R_{c′} one of R_{3′} or R_{6′}; being R_{c″} one of R_{3″} or R_{6″}; being R_{c″} one of R_{″} or R_{6′″}; being R_{c″″} one of R_{3″″} or R_{6″″}), wherein R₁ to R_{6″″} are as defined in the description, and wherein when different radicals R₁ to R_{6″″} are present simultaneously in Formula I they may be identical or different.

Additionally to the above-mentioned substitutions, in connection with cycloalkyl (including alkyl-cycloalkyl), or heterocycly (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with

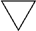

### or ═O.

In connection with cycloalkyl (including alkyl-cycloalkyl), or heterocycly (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with

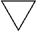

(leading to a spiro structure) or with ═O.

A ring system is a system consisting of at least one ring of connected atoms but including also systems in which two or more rings of connected atoms are joined with “joined” meaning that the respective rings are sharing one (like a spiro structure), two or more atoms being a member or members of both joined rings.

The term “leaving group” means a molecular fragment that departs with a pair of electrons in heterolytic bond cleavage. Leaving groups can be anions or neutral molecules. Common anionic leaving groups are halides such as Cl—, Br—, and I—, and sulfonate esters, such as tosylate (TsO—) or mesylate.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes via ionic interactions. The term “physiologically acceptable salt” means in the context of this invention any salt that is physiologically tolerated (most of the time meaning not being toxic—especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be present in crystalline form or in the form of free compounds like a free base or acid.

Any compound that is a solvate of a compound according to the invention like a compound according to general formula I defined above is understood to be also covered by the scope of the invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent). Especially preferred examples include hydrates and alcoholates, like methanolates or ethanolates.

Any compound that is a prodrug of a compound according to the invention like a compound according to general Formula I defined above is understood to be also covered by the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a N-oxide of a compound according to the invention like a compound according to general formula I defined above is understood to be also covered by the scope of the invention.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or of a nitrogen by ¹⁵N-enriched nitrogen are within the scope of this invention. This would especially also apply to the provisos described above so that any mentioning of hydrogen or any “H” in a formula would also cover deuterium or tritium.

The compounds of formula (I) as well as their salts or solvates of the compounds are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

In a further embodiment the compound according to the invention of general Formula (I) is a compound

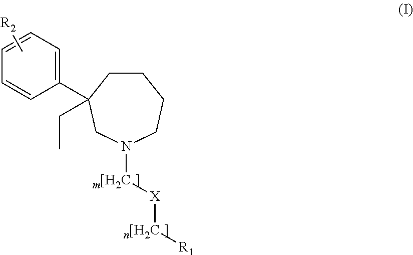

wherein
m is 1, 2 or 3;
n is 0, 1 or 2;
X is selected from a —CH₂N(R_{1′})—, —C(O)N(R_{1′})— and —CH₂O—;
R₁ is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl;
R_{1′} is selected from substituted or unsubstituted C₁₋₆ alkyl, substituted or unsubstituted C₂a alkenyl, substituted or unsubstituted C₂₋₆ alkynyl;
alternatively, when X is —CH₂N(R_{1′})— or —C(O)N(R_{1′})— R₁ and R_{1′} taken together with the connecting N—[CH₂]ₙ atoms may form a substituted or unsubstituted up to 6-member heterocyclyl (meaning also an “up to 6 membered heterocyclic ring”);
wherein the alkyl, alkenyl or alkynyl in R₁ or R_{1′}, if substituted, is substituted with one or more substituents selected from —OR₃, halogen, —CN, haloalkyl, haloalkoxy and —NR₃R_{3′″};
wherein said cycloalkyl, aryl or heterocyclyl in R₁, or said heterocyclyl resulting when X is —CH₂N(R_{1′})— or —C(O)N(R_{1′})—, and R₁ and R_{1′} are taken together (hereinafter named “R₁-R_{1′}”), if substituted, is substituted with one or more substituent/s selected from aryl, halogen, —R₃, —OR₃, —NO₂, —NR₃R_{3′″}, —NR₃C(O)R_{3′}, —NR₃S(O)₂R_{3′}, —S(O)₂NR₃R_{3′}, —NR₃C(O)NR_{3′}R_{3″}, —SR₃, —S(O)R₃, —S(O)₂R₃, —CN, haloalkyl, haloalkoxy, —C(O)OR₃, —C(O)NR₃R_{3′}, —OCH₂CH₂OH, —NR₃S(O)₂NR_{3′}R_{3″} and —C(CH₃)₂OR₃; wherein
R₃, R_{3″} and R_{3″} are independently selected from hydrogen, unsubstituted C₁₋₆ alkyl, unsubstituted C₂₋₆ alkenyl and unsubstituted C₂₋₆ alkynyl;
and wherein R_{3′″} is selected from hydrogen, unsubstituted C₁₋₆ alkyl, unsubstituted C₂₋₆ alkenyl, unsubstituted C₂₋₆ alkynyl and -Boc;
   R₂ is selected from hydrogen, halogen, —R₄, —OR₄, —NO₂, —NR₄R_{4′″}, —NR₄C(O)R_{4′}, —NR₄S(O)₂R_{4′}, —S(O)₂NR₄R_{4′}, —NR₄C(O)NR_{4′}R_{4″}, —SR₄, —S(O)R₄, —S(O)₂R₄, —OS(O)₂R₄, —CN, haloalkyl, haloalkoxy, —C(O)OR₄, —C(O)NR₄R_{4′}, —OCH₂CH₂OH, —NR₄S(O)₂NR_{4′}R_{4″} and —C(CH₃)₂OR₄; wherein
R₄, R_{4′} and R_{4″} are independently selected from hydrogen, unsubstituted C₁₋₆ alkyl, unsubstituted C₂₋₆ alkenyl, and unsubstituted C₂₋₆ alkynyl;
R_{4′″} is selected from hydrogen, unsubstituted C₁₋₆ alkyl, unsubstituted C₂₋₆ alkenyl, unsubstituted C₂₋₆ alkynyl and -Boc;
   wherein the alkyl, alkenyl or alkynyl, other than those defined in R₁ or R_{1′}, if substituted, is substituted with one or more substituents selected from —OR₅, halogen, —CN, haloalkyl, haloalkoxy, —NR₅R_{5′″};
wherein R₅, R_{5′} and R_{5″} are independently selected from hydrogen, unsubstituted C₁₋₅ alkyl, unsubstituted C₂₋₅ alkenyl, and unsubstituted C₂₋₅ alkynyl;
and wherein R_{5′″} is selected from hydrogen, unsubstituted C₁₋₅ alkyl, unsubstituted C₂₋₅ alkenyl, unsubstituted C₂₋₅ alkynyl and -Boc;
   and/or
   wherein the aryl, heterocyclyl or cycloalkyl, other than those defined in R₁, or the heterocyclyl other than those defined in R₁-R_{1″}, if substituted, is substituted with one or more substituents selected from halogen, —R₆, —OR₆, —NO₂, —NR₆R_{6″}, —NR₆C(O)R_{6′}, —NR₆S(O)₂R_{6′}, —S(O)₂NR₆R_{6′}, —NR₆C(O)NR_{6′}R_{6″}, —SR₆, —S(O)R₆, —S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and —C(CH₃)₂OR₆;
wherein R₆, R_{6′} and R_{6″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl, and unsubstituted heterocyclyl;
and wherein R_{6′″} is selected from hydrogen, unsubstituted C₁₋₆ alkyl, unsubstituted C₂₋₆ alkenyl, unsubstituted C₂₋₆ alkynyl and -Boc;

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 1, 2 or 3;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

n is 0, 1 or 2;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is selected from a —CH₂N(R_{1′})—, —C(O)N(R_{1′}) and —CH₂O—;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

X is a —CH₂N(R_{1′})—;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

### X is —C(O)N(R_{1′})—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

### X is —CH₂O—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, and substituted or unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl and substituted or unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from hydrogen, substituted or unsubstituted C₁₋₆ alkyl, substituted or unsubstituted cycloalkyl and substituted or unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{1′} is selected from substituted or unsubstituted C₁₋₆ alkyl, substituted or unsubstituted C₂₋₆ alkenyl, and substituted or unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{1′} is substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ and R_{1′} taken together with the connecting N—[CH₂]ₙ atoms may form a substituted or unsubstituted up to 6-member heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ and R_{1′} taken together with the connecting N—[CH₂]ₙ atoms may form a substituted or unsubstituted 6-member heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ and R_{1′} taken together with the connecting N atom may form a substituted or unsubstituted up to 6-member heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ and R_{1′} taken together with the connecting N atom may form a substituted or unsubstituted 6-member heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{1″} being selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, and substituted or unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{1″} being selected from hydrogen, substituted or unsubstituted C₁₋₆ alkyl, substituted or unsubstituted C₂₋₆ alkenyl, or substituted or unsubstituted C₂₋₆ alkynyl and substituted or unsubstituted aryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R_{1″} being selected from hydrogen, substituted or unsubstituted C₁₋₆ alkyl and substituted or unsubstituted aryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from hydrogen, halogen, —R₄, —OR₄, —NO₂, —NR₄R_{4′″}, —NR₄C(O)R_{4′}, —NR₄S(O)₂R_{4′}, —S(O)₂NR₄R_{4′}, —NR₄C(O)NR_{4′}R_{4″}, —SR₄, —S(O)R₄, —S(O)₂R₄, —OS(O)₂R₄, —CN, haloalkyl, haloalkoxy, —C(O)OR₄, —C(O)NR₄R_{4′}, —OCH₂CH₂OH, —NR₄S(O)₂NR₄R_{4″} and —C(CH₃)₂OR₄;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is —OR₄,
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃, R_{3′} and R_{3″} are independently selected from hydrogen, unsubstituted C₁₋₆ alkyl, unsubstituted C₂₋₆ alkenyl and unsubstituted C₂₋₆ alkynyl;
and wherein R_{3′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃ is selected from hydrogen, unsubstituted C₁₋₆ alkyl, unsubstituted C₂₋₆ alkenyl and unsubstituted C₂₋₆ alkynyl;
and wherein R_{3′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃, R_{3′} and R_{3″} are independently selected from hydrogen, unsubstituted C₁₋₆ alkyl, unsubstituted C₂₋₆ alkenyl and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₃, R_{3′}, and R_{3″} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R_{3′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄, R_{4′} and R_{4″} are independently selected from hydrogen, unsubstituted C₁₋₆ alkyl, unsubstituted C₂₋₆ alkenyl, and unsubstituted C₂₋₆ alkynyl;
R_{4′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄, R_{4′} and R_{4″} are independently selected from hydrogen, unsubstituted C₁₋₆ alkyl, unsubstituted C₂₋₆ alkenyl, and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄, R_{4′} and R_{4″} are independently selected from hydrogen and unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₄, is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₅, R_{5′} and R_{5″} are independently selected from hydrogen, unsubstituted C₁₋₅ alkyl, unsubstituted C₂₋₅ alkenyl, and unsubstituted C₂₋₅ alkynyl;
and wherein R_{5′″} is selected from hydrogen, unsubstituted C₁₋₅ alkyl, unsubstituted C₂₋₅ alkenyl, unsubstituted C₂₋₅ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₅, R_{5′} and R_{5″} are independently selected from hydrogen, unsubstituted C₁₋₅ alkyl, unsubstituted C₂₋₅ alkenyl, and unsubstituted C₂₋₅ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{5′″} is selected from hydrogen, unsubstituted C₁₋₅ alkyl, unsubstituted C₂₋₅ alkenyl, unsubstituted C₂₋₅ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₆, R_{6′} and R_{6″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl, and unsubstituted heterocyclyl;
and wherein R_{6′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₆, R_{6′} and R_{6″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl, and unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{6′″} is selected from hydrogen, unsubstituted C₁₋₆ alkyl, unsubstituted C₂₋₆ alkenyl, unsubstituted C₂₋₆ alkynyl and -Boc;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I), is a compound wherein

m is 1, 2 or 3;
n is 0, 1 or 2;
X is selected from a —CH₂N(R_{1′})—, —C(O)N(R_{1′})— and —CH₂O—;
R₁ is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl;
wherein
the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl, ethyl, isopropyl or isobutyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the aryl is selected from phenyl, naphtyl, or anthracene; preferably is napthyl and phenyl;
and/or
the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline, more preferably the heterocycle is pyridine;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably is C₃₋₆ cycloalkyl like cyclopropyl, cyclobutyl, cyclopentyl or cyclohexyl, more preferably the cycloalkyl is cyclopropyl;
and/or
R_{1′} is selected from substituted or unsubstituted C₁₋₆ alkyl, substituted or unsubstituted C₂₋₆ alkenyl, substituted or unsubstituted C₂₋₆ alkynyl;
wherein
the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
   R₁ and R_{1′} taken together with the connecting N—[CH₂]ₙ atoms may form a substituted or unsubstituted up to 6-member heterocyclyl; preferably may form a substituted or unsubstituted 6-member heterocyclyl;
   wherein
   the heterocyclyl is a heterocyclic ring system of one saturated or unsaturated ring which contains one or more heteroatoms from the group consisting of nitrogen, oxygen and/or sulfur in the ring; preferably is a heterocyclic ring system of one saturated or unsaturated ring which contains one or more heteroatoms from the group consisting of nitrogen, oxygen and/or sulfur in the ring, more preferably is selected from imidazole, oxadiazole, tetrazole, pyridine, pyrimidine, piperidine, piperazine, thiazole, tetrahydropyrane, morpholine, furan, triazole, isoxazole, pyrazole, thiophene, pyrrole, pyrazine, oxopyrrolidine and pyrimidine, more preferably the heterocycle is piperidine, piperazine or morpholine;
and/or
   R₁ and R_{1′} taken together with the connecting N atom may form a substituted or unsubstituted up to 6-member heterocyclyl; preferably may form a substituted or unsubstituted 6-member heterocyclyl;
   wherein
   the heterocyclyl is a heterocyclic ring system of one saturated or unsaturated ring which contains one or more heteroatoms from the group consisting of nitrogen, oxygen and/or sulfur in the ring; preferably is a heterocyclic ring system of one saturated or unsaturated rings which contains one or more heteroatoms from the group consisting of nitrogen, oxygen and/or sulfur in the ring, more preferably is selected from imidazole, oxadiazole, tetrazole, pyridine, pyrimidine, piperidine, piperazine, thiazole, tetrahydropyrane, morpholine, furan, triazole, isoxazole, pyrazole, thiophene, pyrrole, pyrazine, oxopyrrolidine and pyrimidine, more preferably the heterocycle is piperidine, piperazine or morpholine;
   and/or
   R_{1″} being selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl;
   wherein
   the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl;
   and/or
   the C₂₋₆-alkenyl is preferably selected from ethylene, propylene, butylene, pentylene, hexylene, isopropylene and isobutylene;
   and/or
   the C₂₋₆-alkynyl is preferably selected from ethyne, propyne, butyne, pentyne, hexyne, isopropyne and isobutyne;
   and/or
   the aryl is selected from phenyl, naphtyl, or anthracene; preferably is napthyl and phenyl; more preferably the aryl is phenyl;
   and/or
   the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
   and/or
   the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably is C₃₋₆ cycloalkyl like cyclopropyl, cyclobutyl, cyclopentyl or cyclohexyl;
   and/or
   R₂ is selected from hydrogen, halogen, —R₄, —OR₄, —NO₂, —NR₄R_{4″}, NR₄C(O)R_{4′}, —NR₄S(O)₂R_{4′}, —S(O)₂NR₄R_{4′}, —NR₄C(O)NR_{4′}R_{4″}, —SR₄, —S(O)R₄, —S(O)₂R₄, —OS(O)₂R₄, —CN, haloalkyl, haloalkoxy, —C(O)OR₄, —C(O)NR₄R_{4′}, —OCH₂CH₂OH, —NR₄S(O)₂NR_{4′}R_{4″} and —C(CH₃)₂OR₄;
   wherein
   the alkyl is preferably selected from methyl, ethyl, propyl, butyl, pentyl, hexyl, isopropyl and 2-methylpropyl;
and/or
   R₃, R_{3″} and R_{3″} are independently selected from hydrogen, unsubstituted C₁₋₆ alkyl, unsubstituted C₂₋₆ alkenyl and unsubstituted C₂₋₆ alkynyl;
   wherein
   the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl;
   the C₂₋₆-alkenyl is preferably selected from ethylene, propylene, butylene, pentylene, hexylene, isopropylene and isobutylene;
   and/or
   the C₂₋₆-alkynyl is preferably selected from ethyne, propyne, butyne, pentyne, hexyne, isopropyne and isobutyne;
   and/or
   R_{3′″} is selected from hydrogen, unsubstituted C₁₋₅ alkyl, unsubstituted C₂₋₆ alkenyl, unsubstituted C₂₋₆ alkynyl and -Boc;
   wherein
   the C₁₋₆ alkyl is preferably selected from methyl, ethyl, propyl, butyl, pentyl, hexyl, isopropyl, isobutyl and 2-methylpropyl;
   and/or
   the C₂₋₆-alkenyl is preferably selected from ethylene, propylene, butylene, pentylene, hexylene, isopropylene and isobutylene;
   and/or
   the C₂₋₆-alkynyl is preferably selected from ethyne, propyne, butyne, pentyne, hexyne, isopropyne and isobutyne;
   R₄, R_{4′} and R_{4″} are independently selected from hydrogen, unsubstituted C₁₋₆ alkyl, unsubstituted C₂₋₆ alkenyl, and unsubstituted C₂₋₆ alkynyl;
   wherein
   the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl;
   and/or
   the C₂₋₆-alkenyl is preferably selected from ethylene, propylene, butylene, pentylene, hexylene, isopropylene and isobutylene;
   and/or
   the C₂₋₆-alkynyl is preferably selected from ethyne, propyne, butyne, pentyne, hexyne, isopropyne and isobutyne;
and/or
   R_{4′″} is selected from hydrogen, unsubstituted C₁₋₆ alkyl, unsubstituted C₂₋₆ alkenyl, unsubstituted C₂₋₆ alkynyl and -Boc;
   wherein
   the C₂₋₆ alkyl is preferably selected from methyl, ethyl, propyl, butyl, pentyl, hexyl, isopropyl, isobutyl and 2-methylpropyl;
   and/or
   the C₂₋₆-alkenyl is preferably selected from ethylene, propylene, butylene, pentylene, hexylene, isopropylene and isobutylene;
   and/or
   the C₂₋₆-alkynyl is preferably selected from ethyne, propyne, butyne, pentyne, hexyne, isopropyne and isobutyne;
and/or
   R₅, R_{5′} and R_{5″} are independently selected from hydrogen, unsubstituted C₁₋₅ alkyl, unsubstituted C₂₋₅ alkenyl, and unsubstituted C₂₋₅ alkynyl;
   wherein
   the C₁₋₆ alkyl is preferably selected from methyl, ethyl, propyl, butyl, pentyl, hexyl, isopropyl, isobutyl and 2-methylpropyl;
   and/or
   the C₂₋₆-alkenyl is preferably selected from ethylene, propylene, butylene, pentylene, hexylene, isopropylene and isobutylene;
   and/or
   the C₂₋₆-alkynyl is preferably selected from ethyne, propyne, butyne, pentyne, hexyne, isopropyne and isobutyne;
   and/or
   R_{5′″} is selected from hydrogen, unsubstituted C₁₋₅ alkyl, unsubstituted C₂₋₅ alkenyl, unsubstituted C₂₋₅ alkynyl and -Boc;
   wherein
   the C₁₋₆ alkyl is preferably selected from methyl, ethyl, propyl, butyl, pentyl, hexyl, isopropyl, isobutyl and 2-methylpropyl;
   and/or
   the C₂₋₆-alkenyl is preferably selected from ethylene, propylene, butylene, pentylene, hexylene, isopropylene and isobutylene;
   and/or
   the C₂₋₆-alkynyl is preferably selected from ethyne, propyne, butyne, pentyne, hexyne, isopropyne and isobutyne;
   and/or
   R₆, R_{6′} and R_{6″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl, and unsubstituted heterocyclyl;
   wherein
   the C₁₋₆ alkyl is preferably selected from methyl, ethyl, propyl, butyl, pentyl, hexyl, isopropyl, isobutyl and 2-methylpropyl;
   and/or
   the C₂₋₆-alkenyl is preferably selected from ethylene, propylene, butylene, pentylene, hexylene, isopropylene and isobutylene;
   and/or
   the C₂₋₆-alkynyl is preferably selected from ethyne, propyne, butyne, pentyne, hexyne, isopropyne and isobutyne;
   and/or
   the aryl is selected from phenyl, naphtyl, or anthracene; preferably is napthyl and phenyl;
   and/or
   the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably is C₃₋₆ cycloalkyl like cyclopropyl, cyclobutyl, cyclopentyl or cyclohexyl;
   and/or
   R_{6′″} is selected from hydrogen, unsubstituted C₁₋₆ alkyl, unsubstituted C₂₋₆ alkenyl, unsubstituted C₂₋₆ alkynyl and -Boc;
   wherein
   the C₁₋₆ alkyl is preferably selected from methyl, ethyl, propyl, butyl, pentyl, hexyl, isopropyl, isobutyl and 2-methylpropyl;
   and/or
   the C₂₋₆-alkenyl is preferably selected from ethylene, propylene, butylene, pentylene, hexylene, isopropylene and isobutylene;
   and/or
   the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁ as defined in any of the embodiments of the present invention,

the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl, ethyl, isopropyl or isobutyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the aryl is selected from phenyl, naphtyl, or anthracene; preferably is napthyl and phenyl;
and/or
the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline, more preferably the heterocycle is pyridine;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably is C₃₋₆ cycloalkyl like cyclopropyl, cyclobutyl, cyclopentyl or cyclohexyl, more preferably the cycloalkyl is cyclopropyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{1′} as defined in any of the embodiments of the present invention, the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl;

and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁,R_{1′} as defined in any of the embodiments of the present invention,

the heterocyclyl is a heterocyclic ring system of one saturated or unsaturated ring which contains one or more heteroatoms from the group consisting of nitrogen, oxygen and/or sulfur in the ring; preferably is a heterocyclic ring system of one saturated or unsaturated rings which contains one or more heteroatoms from the group consisting of nitrogen, oxygen and/or sulfur in the ring, more preferably is selected from imidazole, oxadiazole, tetrazole, pyridine, pyrimidine, piperidine, piperazine, thiazole, tetrahydropyrane, morpholine, furan, triazole, isoxazole, pyrazole, thiophene, pyrrole, pyrazine, oxopyrrolidine and pyrimidine, more preferably the heterocycle is piperidine, piperazine or morpholine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁ as defined in any of the embodiments of the present invention,

wherein
the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the aryl is selected from phenyl, naphtyl, or anthracene; preferably is napthyl and phenyl; more preferably the aryl is phenyl;
and/or
the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably is C₃₋₆ cycloalkyl like cyclopropyl, cyclobutyl, cyclopentyl or cyclohexyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₂ as defined in any of the embodiments of the present invention,

the alkyl is preferably selected from methyl, ethyl, propyl, butyl, pentyl, hexyl, isopropyl, isobutyl and 2-methylpropyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₃, R_{3′} and R_{3″} as defined in any of the embodiments of the present invention,

the C₁₋₆ alkyl is preferably selected from methyl, ethyl, propyl, butyl, pentyl, hexyl, isopropyl, isobutyl and 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{3″} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₄, R_{4′} and R_{4″} as defined in any of the embodiments of the present invention,

the C₁₋₆ alkyl is preferably selected from methyl, ethyl, propyl, butyl, pentyl, hexyl, isopropyl, isobutyl and 2-methylpropyl; preferably, C₁₋₆ alkyl is methyl or isopropyl;
and/or
the C₁₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{4′″} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₅, R_{5′} and R_{5″} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{5′″} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₆, R_{6′} and R_{6″} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the aryl is selected from phenyl, naphtyl, or anthracene; preferably is napthyl and phenyl;
and/or
the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from imidazole, oxadiazole, tetrazole, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably is C₃₋₆ cycloalkyl like cyclopropyl, cyclobutyl, cyclopentyl or cyclohexyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{6′″} as defined in any of the embodiments of the present invention,

the C₁₋₆ alkyl is preferably selected from methyl, ethyl, propyl, butyl, pentyl, hexyl, isopropyl, isobutyl and 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 1, 2 or 3; preferably m is 1 or 2;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

n is 0, 1 or 2; preferably n is 0;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

X is selected from a —CH₂N(R_{1′})—, —C(O)N(R_{1′})— and —CH₂O—;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I), the compound is a compound of Formula (I′)

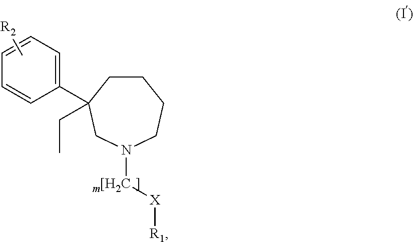

wherein
m is 1, 2 or 3;
X is selected from a —CH₂N(R_{1′})—, —C(O)N(R_{1′})— and —CH₂O—;
R₁ is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl;
R_{1′} is selected from substituted or unsubstituted C₁₋₆ alkyl, substituted or unsubstituted C₂₋₆ alkenyl, substituted or unsubstituted C₂₋₆ alkynyl;
alternatively, when X is —CH₂N(R_{1′})— or —C(O)N(R_{1′})—, R₁ and R_{1′} taken together with the connecting N atom may form a substituted or unsubstituted up to 6-member heterocyclyl;
wherein the alkyl, alkenyl or alkynyl in R₁ or R_{1′}, if substituted, is substituted with one or more substituents selected from —OR₃, halogen, —CN, haloalkyl, haloalkoxy and —NR₃R_{3′″};
wherein said cycloalkyl, aryl or heterocyclyl in R₁, or said heterocyclyl in R₁-R_{1′}, if substituted, is substituted with one or more substituent/s selected from aryl, halogen, —R₃, —OR₃, —NO₂, —NR₃R_{3′″}, —NR₃C(O)R_{3′}, —NR₃S(O)₂R_{3′}, —S(O)₂NR₃R_{3′}, —NR₃C(O)NR₃R_{3″}, —SR₃, —S(O)R₃, —S(O)₂R₃, —CN, haloalkyl, haloalkoxy, —C(O)OR₃, —C(O)NR₃R_{3′}, —OCH₂CH₂OH, —NR₃S(O)₂NR_{3′}R_{3″} and —C(CH₃)₂OR₃; wherein
R₃, R_{3′} and R_{3″} are independently selected from hydrogen, unsubstituted C₁₋₆ alkyl, unsubstituted C₂₋₆ alkenyl and unsubstituted C₂₋₆ alkynyl;
and wherein R_{3′″} is selected from hydrogen, unsubstituted C₁₋₆ alkyl, unsubstituted C₂₋₆ alkenyl, unsubstituted C₂₋₆ alkynyl and -Boc;
   R₂ is selected from hydrogen, halogen, —R₄, —OR₄, —NO₂, —NR₄R_{4′″}, —NR₄C(O)R_{4′}, —NR₄S(O)₂R_{4′}, —S(O)₂NR₄R_{4′}, —NR₄C(O)NR₄R_{4″}, —SR₄, —S(O)R₄, —S(O)₂R₄, —OS(O)₂R₄, —CN, haloalkyl, haloalkoxy, —C(O)OR₄, —C(O)NR₄R_{4′}, —OCH₂CH₂OH, —NR₄S(O)₂NR_{4′}R_{4″} and —C(CH₃)₂OR₄; wherein
R₄, R_{4′} and R_{4″} are independently selected from hydrogen, unsubstituted C₁₋₆ alkyl, unsubstituted C₂₋₆ alkenyl, and unsubstituted C₂₋₆ alkynyl;
R_{4′″} is selected from hydrogen, unsubstituted C₁₋₆ alkyl, unsubstituted C₂₋₆ alkenyl, unsubstituted C₂₋₆ alkynyl and -Boc;
   wherein the alkyl, alkenyl or alkynyl, other than those defined in R₁ or R_{1′}, if substituted, is substituted with one or more substituents selected from —OR₅, halogen, —CN, haloalkyl, haloalkoxy, —NR₅R_{5″′};
wherein R₅, R_{5′} and R_{5″} are independently selected from hydrogen, unsubstituted C₁₋₅ alkyl, unsubstituted C₂₋₅ alkenyl, and unsubstituted C₂₋₅ alkynyl;
and wherein R_{5″′} is selected from hydrogen, unsubstituted Cis alkyl, unsubstituted C₂₋₅ alkenyl, unsubstituted C₂₋₅ alkynyl and -Boc;
   and/or
   wherein the aryl, heterocyclyl or cycloalkyl, other than those defined in R₁, or the heterocyclyl other than those defined in R₁-R_{1′}, if substituted, is substituted with one or more substituents selected from halogen, —R₆, —OR₆, —NO₂, —NR₆R_{6″}, —NR₆C(O)R_{6′}, —NR₆S(O)₂R_{6′}, —S(O)₂NR₆R_{6′}, —NR₆C(O)NR_{6′}R_{6″}, —SR₆, —S(O)R₆, —S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and —C(CH₃)₂OR_{6′};
wherein R₆, R_{6′} and R_{6″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl, and unsubstituted heterocyclyl;
and wherein R_{6′″} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further preferred embodiment of the invention according to general Formula (I) the compound is a compound, wherein

X is selected from a —CH₂N(R₁)—, —C(O)N(R_{1′})— and —CH₂O—; and/or
m is 1, 2 or 3; preferably m is 1 or 2; and/or
n is 0, 1 or 2; preferably n is 0;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further preferred embodiment of the invention according to general Formula (I) the compound is a compound of Formula (I),

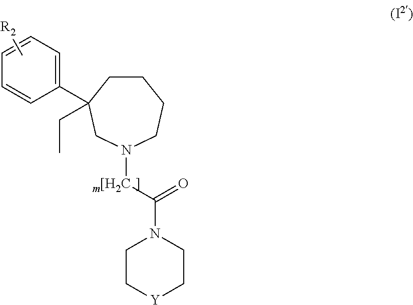

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further preferred embodiment of the invention according to general Formula (I) the compound is a compound of Formula (I^{2′}),

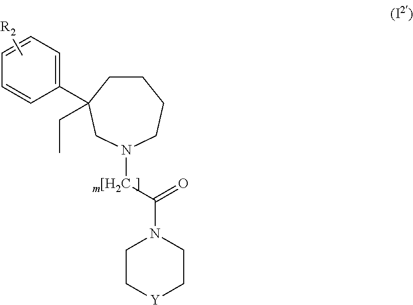

with Y being selected from —O—, —N(R_{1″})— or —CH(R_{1″})— and
R_{1″} being selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl; preferably being selected from hydrogen, unsubstituted C₁₋₄ alkyl, or unsubstituted aryl,
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further preferred embodiment of the invention according to general Formula (I) the compound is a compound of Formula (I^{3′}),

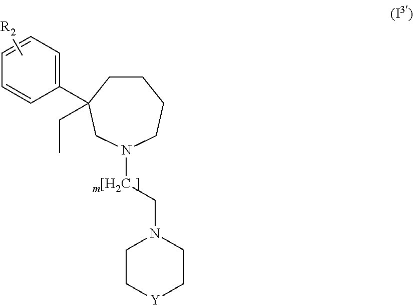

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further preferred embodiment of the invention according to general Formula (I) the compound is a compound of Formula (I^{3′}),

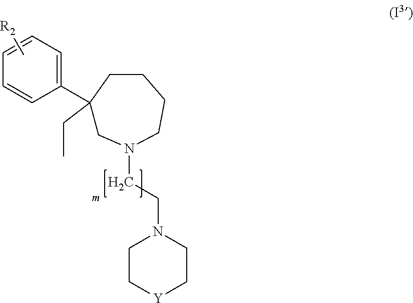

with Y being selected from —O—, —N(R_{1″})— or —CH(R_{1″})— and
R_{1′} being selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl; preferably being selected from hydrogen, unsubstituted C₁₋₄ alkyl, or unsubstituted aryl,
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further preferred embodiment of the invention according to general Formula (I) the compound is a compound of Formula (I^{4′}),

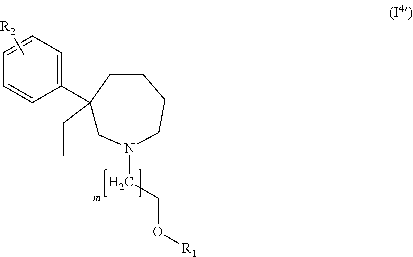

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment

R₁ is hydrogen or a substituted or unsubstituted group selected from methyl, ethyl, isopropyl, isobutyl, cyclopropyl and pyridine.

In a preferred embodiment

R_{1′} is substituted or unsubstituted methyl, more preferably R_{1′} is unsubstituted methyl.

In a preferred embodiment

R₁-R_{1′} is a substituted or unsubstituted group selected from piperazine, piperidine and morpholine.

In a preferred embodiment

R_{1″} is hydrogen or a substituted or unsubstituted group selected from methyl and phenyl, more preferably is hydrogen or a unsubstituted group selected from methyl and phenyl,

In a preferred embodiment

R₂ is —OH, unsubstituted —O-methyl, —NH₂, —NHS(O)₂-methyl or —NHS(O)₂-isopropyl.

In a preferred embodiment

R₂ is hydrogen or —OR₄, preferably —OH or —O-methyl, more preferably in meta position.

In a preferred embodiment

R₂ is hydrogen or —OR₄.

In a more preferred embodiment

R₂ is hydrogen or —OR₄ in meta position.

In a more preferred embodiment

### R₂ is —OH or —O-methyl.

In a most preferred embodiment

R₂ is —OH or —O-methyl in meta position.

In a preferred embodiment

R₃ is hydrogen, unsubstituted ethyl or unsubstituted phenyl.

In a preferred embodiment

R₄ is hydrogen or unsubstituted methyl.

In a preferred embodiment

R_{4′} is unsubstituted methyl or unsubstituted isopropyl.

In a preferred embodiment

R_{4′″} is hydrogen.

In a preferred embodiment

R₄ is hydrogen while R_{4′} is unsubstituted methyl or unsubstituted isopropyl.

In a preferred embodiment

R₄ and R_{4′″} are both hydrogen.

In another preferred embodiment

n is 0;

In another preferred embodiment

m is 1 or 2;

In an particular embodiment

the halogen is fluorine or chlorine, preferably fluorine.

In a preferred further embodiment, the compounds of the general Formula (I) are selected from

| EX | Chemical name |
|---|---|
| 1 | 3-ethyl-3-(3-methoxyphenyl)-1-(2-(4-methylpiperazin-1- |
| | yl)ethyl)azepane |
| 2 | 3-ethyl-3-(3-methoxyphenyl)-1-(2-(4-phenylpiperazin-1- |
| | yl)ethyl)azepane |
| 3 | 4-(2-(3-Ethyl-3-(3-methoxyphenyl)azepan-1-yl)ethyl)morpholine |
| 4 | 3-ethyl-3-(3-methoxyphenyl)-1-(2-(piperidin-1-yl)ethyl)azepane |
| 5 | 1-(2-ethoxyethyl)-3-ethyl-3-(3-methoxyphenyl)azepane |
| 6 | 3-(3-ethyl-1-(2-(4-methylpiperazin-1-yl)ethyl)azepan-3-yl)phenol |
| 7 | 3-(3-ethyl-1-(2-(4-phenylpiperazin-1-yl)ethyl)azepan-3-yl)phenol |
| 8 | 3-(3-ethyl-1-(2-morpholinoethyl)azepan-3-yl)phenol |
| 9 | 3-(3-ethyl-1-(2-(piperidin-1-yl)ethyl)azepan-3-yl)phenol |
| 10 | 3-(3-ethyl-1-(2-hydroxyethyl)azepan-3-yl)phenol |
| 11 | 3-(1-(2-ethoxyethyl)-3-ethylazepan-3-yl)phenol |
| 12 | 3-(3-ethyl-1-(2-isopropoxyethyl)azepan-3-yl)phenol |
| 13 | 2-(3-ethyl-3-(3-hydroxyphenyl)azepan-1-yl)-1-(piperidin- |
| | 1-yl)ethanone |
| 14 | 3-(3-ethy-1-(2-(pyridin-3-yloxy)ethyl)azepan-3-yl)phenol |
| 15 | 2-(3-ethyl-3-(3-hydroxyphenyl)azepan-1-yl)-N,N- |
| | dimethylacetamide |
| 16 | 2-(3-ethyl-3-(3-hydroxyphenyl)azepan-1-yl)-1-morpholinoethanone |
| 17 | 3-(3-ethyl-1-(2-methoxyethyl)azepan-3-yl)phenol |
| 18 | 3-(1-(2-cyclopropoxyethyl)-3-ethylazepan-3-yl)phenol |
| 19 | 3-(3-ethyl-1-(2-(2-hydroxy-2-methylpropoxy)ethyl)azepan- |
| | 3-yl)phenol |
| 20 | (S)-3-(1-(2-ethoxyethyl)-3-ethylazepan-3-yl)phenol |
| 21 | (R)-3-(1-(2-ethoxyethyl)-3-ethylazepan-3-yl)phenol |
| 22 | 3-[1-(2-Ethoxyethyl)-3-ethylazepan-3-yl]aniline |
| 23 | N-{3-[1-(2-Ethoxyethyl)-3-ethylazepan-3- |
| | yl]pheny}methanesulfonamide |
| 24 | N-{3-[1-(2-Ethoxyethyl)-3-ethylazepan-3-yl]phenyl}propane-2- |
| | sulfonamide |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl;
R_{1′} is selected from substituted or unsubstituted C₁₋₆ alkyl, substituted or unsubstituted C₂₋₆ alkenyl, substituted or unsubstituted C₂₋₆ alkynyl;
alternatively, when X is —CH₂N(R_{1′})— or —C(O)N(R_{1′})—, R₁ and R_{1′} taken together with the connecting N—[CH₂]ₙ atoms may form a substituted or unsubstituted up to 6-member heterocyclyl;
wherein the alkyl, alkenyl or alkynyl in R₁ or R_{1′}, if substituted, is substituted with one or more substituents selected from —OR₃, halogen, —CN, haloalkyl, haloalkoxy and —NR₃R_{3′″};
wherein said cycloalkyl, aryl or heterocycly in R₁, or said heterocyclyl in R₁-R_{1′}, if substituted, is substituted with one or more substituent/s selected from aryl, halogen, —R₃, —OR₃, —NO₂, —NR₃R_{3′″}, —NR₃C(O)R_{3′}, —NR₃S(O)₂R_{3′}, —S(O)₂NR₃R_{3′}, —NR₃C(O)NR_{3′}R_{3″}, —SR₃, —S(O)R₃, —S(O)₂R₃, —CN, haloalkyl, haloalkoxy, —C(O)OR₃, —C(O)NR₃R_{3′}, —OCH₂CH₂OH, —NR₃S(O)₂NR_{3′}R_{3″} and —C(CH₃)₂OR₃; wherein
R₃, R_{3′} and R_{3′″} are independently selected from hydrogen, unsubstituted C₁₋₆ alkyl, unsubstituted C₂₋₆ alkenyl and unsubstituted C₂₋₆ alkynyl;
and wherein R_{3′″} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I′),

R₁ is selected from hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocycyl;
R_{1′} is selected from substituted or unsubstituted C₁₋₆ alkyl, substituted or unsubstituted C₂₋₆ alkenyl, substituted or unsubstituted C₂₋₆ alkynyl;
alternatively, when X is —CH₂N(R_{1′})— or —C(O)N(R_{1′})—. R₁ and R_{1′} taken together with the connecting N atom may form a substituted or unsubstituted up to 6-member heterocyclyl;
wherein the alkyl, alkenyl or alkynyl in R₁ or R_{1′} if substituted, is substituted with one or more substituents selected from —OR₃, halogen, —CN, haloalkyl, haloalkoxy and —NR₃R_{3′″};
wherein said cycloalkyl, aryl or heterocyclyl in R₁, or said heterocyclyl in R₁-R_{1′}, if substituted, is substituted with one or more substituent/s selected from aryl, halogen, —R₃, —OR₃, —NO₂, —NR₃R_{3′″}, —NR₃C(O)R₃, —NR₃S(O)₂R_{3′}, —S(O)₂NR₃R_{3′}, —NR₃C(O)NR_{3′}R_{3″}, —SR₃, —S(O)R₃, —S(O)₂R₃, —CN, haloalkyl, haloalkoxy, —C(O)OR₃, —C(O)NR₃R_{3′}, —OCH₂CH₂OH, —NR₃S(O)₂NR_{3′}R_{3″} and —C(CH₃)₂OR₃; wherein
R₃, R_{3′} and R_{3″} are independently selected from hydrogen, unsubstituted C₁₋₆ alkyl, unsubstituted C₂₋₆ alkenyl and unsubstituted C₂₋₆ alkynyl;
and wherein R_{3′″} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention, in the compound of general Formula (I),

the alkyl, alkenyl or alkynyl, other than those defined in R₁ or R_{1′}, if substituted, is substituted with one or more substituents selected from —OR₅, halogen, —CN, haloalkyl, haloalkoxy, —NR₅R_{5′″};
wherein R₅, R_{5′} and R_{5″} are independently selected from hydrogen, unsubstituted C₁₋₅ alkyl, unsubstituted C₂₋₅ alkenyl, and unsubstituted C₂₋₅ alkynyl;
and wherein R_{5′″} is selected from hydrogen, unsubstituted C₁₋₅ alkyl, unsubstituted C₂₋₅ alkenyl, unsubstituted C₂₋₅ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention, in the compound of general Formula (I),

the aryl, heterocyclyl or cycloalkyl, other than those defined in R₁ or the heterocyclyl other than those defined in R₁-R₁, if substituted, is substituted with one or more substituents selected from halogen, —R₆, —OR₆, —NO₂, —NR₆R_{6″}, —NR₆C(O)R_{6′}, —NR₆S(O)₂R_{6′}, —S(O)₂NR_{6′}, —NR₆C(O)NR_{6′}R_{6″}, —SR₆, —S(O)R₆, —S(O)₂R₆, —CN, haloalkyl, haloalkoxy, —C(O)OR₆, —C(O)NR₆R_{6′}, —OCH₂CH₂OH, —NR₆S(O)₂NR_{6′}R_{6″} and —C(CH₃)₂OR₆;
wherein R₆, R_{6′} and R_{6″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl, and unsubstituted heterocyclyl;
and wherein R_{6′″} is selected from hydrogen, unsubstituted C₁₋₆ alkyl, unsubstituted C₂₋₆ alkenyl, unsubstituted C₂₋₆ alkynyl and -Boc;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention, the alkyl, alkenyl or alkynyl in R₁ or R_{1′}, if substituted, is substituted with one or more substituents selected from —OR₃, halogen, —CN, haloalkyl, haloalkoxy and —NR₃R_{3′″};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) and in relation to R₁ of any of the embodiments of the present invention,
the cycloalkyl, aryl or heterocyclyl in R₁, or said heterocyclyl in R₁-R_{1′}, if substituted, is substituted with one or more substituent/s selected from aryl, halogen, —R₃, —OR₃, —NO₂, —NR₃R_{3′″}, —NR₃C(O)R_{3′}, —NR₃S(O)₂R_{3′}, —S(O)₂NR₃R_{3′}, —NR₃C(O)NR_{3′}R_{3″}, —SR₃, —S(O)R₃, —S(O)₂R₃, —CN, haloalkyl, haloalkoxy, —C(O)OR₃, —C(O)NR₃R_{3′}, —OCH₂CH₂OH, —NR₃S(O)₂NR_{3′}R_{3″} and —C(CH₃)₂OR₃;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I) of any of the embodiments of the present invention,
the cycloalkyl or non-aromatic heterocyclyl in R₁ or in R₁-R_{1′}, if substituted, may also be substituted with

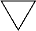

### or ═O;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the halogen is fluorine, chlorine, iodine or bromine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a most preferred embodiment of the compound according to the invention of general Formula (I)

the halogen is fluorine or chlorine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the haloalkyl is —CF₃;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the compound according to the invention of general Formula (I),

the haloalkoxy is —OCF₃;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

As this invention is aimed at providing a compound or a chemically related series of compounds which act as dual ligands of the or receptor and the μ-opioid receptor it is a very preferred embodiment in which the compounds are selected which act as dual ligands of the σ₁ receptor and the μ-opioid receptor and especially compounds which have a binding expressed as Kᵢ which is preferably <1000 nM for both receptors, more preferably <500 nM, even more preferably <100 nM.

In the following the phrase “compound of the invention” is used. This is to be understood as any compound according to the invention as described above according to general Formula (I), (I′), (I²′)), (I³′) or (I^{4′}).

The compounds of the invention represented by the above described Formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

In general the processes are described below in the experimental part. The starting materials are commercially available or can be prepared by conventional methods.

A preferred aspect of the invention is also a process for the production of a compound according to Formula (I), following scheme 1.

A preferred embodiment of the invention is a process for the production of a compound according to Formula (I), wherein R₁, R_{1′}, R_{1″}, R₂, R₃, R_{3′}, R_{3″}, R_{3′″}, R₄, R_{4′}, R_{4″}, R_{4′″}, R₅, R_{5′}, R_{5″}, R_{5′″}, R₆, R_{6′}, R_{6″}, R_{6′″}, X, m and n are as defined in the description, following scheme 1.

In all processes and uses described underneath and in scheme 1 or scheme 1′, the values of R₁, R_{1′}, R_{1″}, R₂, R₃, R_{3′}, R_{3″}, R_{3′″}, R₄, R_{4′}, R_{4″}, R_{4′″}, R₅, R_{5′}, R_{5″}, R_{5′″}, R₆, R_{6′}, R_{6″}, R_{6′″}, X, m and n are as defined in the description, where, L is a leaving group such as halogen, mesylate, tosylate or triflate and Z is chloro, bromo or iodine.

In a particular embodiment there is a process for the production of a compound of Formula (I),

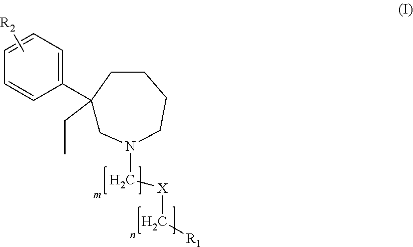

said process comprises the reduction of a compound of Formula XI

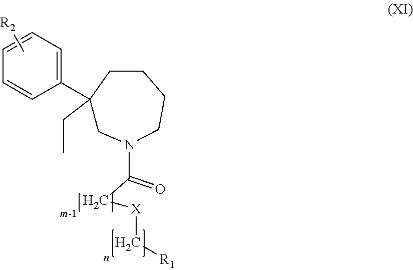

In a particular embodiment there is a process for the production of a compound of Formula (I),

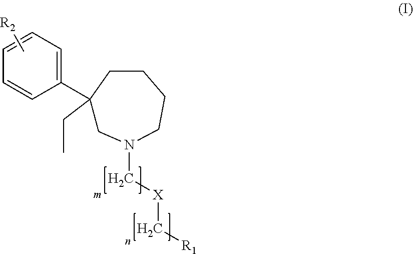

said process comprises the alkylation of a compound of Formula IX

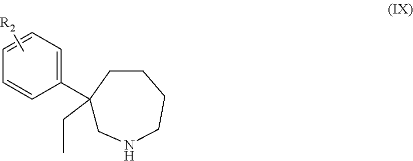

with a compound of formula Xa following STEP 6 of scheme 1

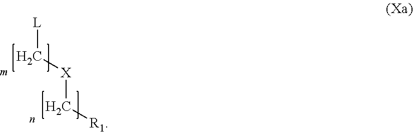

In a particular embodiment there is a process for the production of a compound of Formula (I),

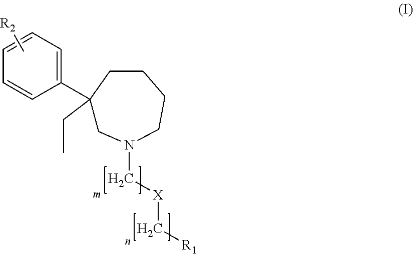

said process comprises the reductive amination of a compound of Formula IX

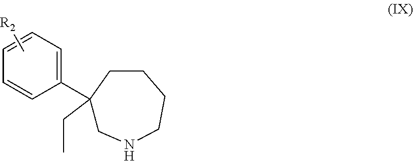

with a compound of formula Xb following STEP 6 of scheme 1

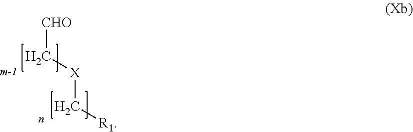

In a particular embodiment there is a process for the production of a compound of Formula (I′),

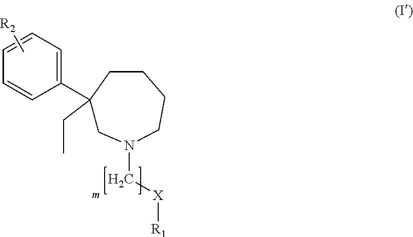

said process comprises the reduction of a compound of Formula XI′

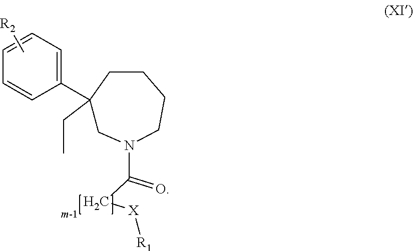

In a particular embodiment there is a process for the production of a compound of Formula (I′),

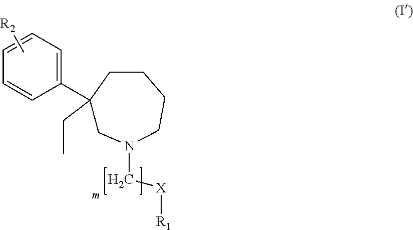

said process comprises the alkylation of a compound of Formula IX

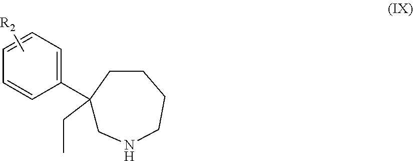

with a compound of formula Xa′ following STEP 6 of scheme 1′

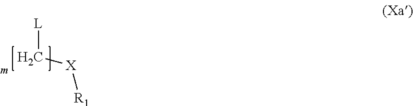

In a particular embodiment there is a process for the production of a compound of Formula (I′),

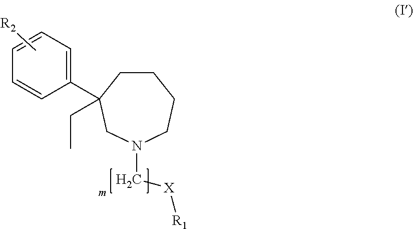

said process comprises the reductive amination of a compound of Formula IX

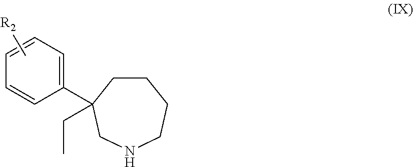

with a compound of formula Xb′ following STEP 6 of scheme 1′

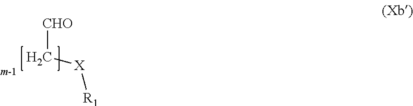

In another particular embodiment a compound of Formula (II),

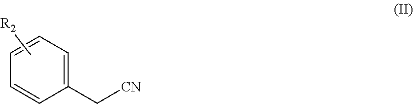

is used for the preparation of a compound of Formula (I).

In another particular embodiment a compound of Formula (III),

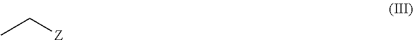

is used for the preparation of a compound of Formula (I).

In another particular embodiment a compound of Formula (IV),

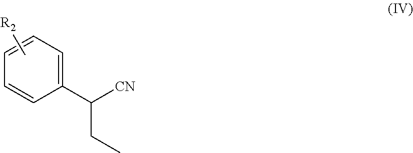

is used for the preparation of a compound of Formula (I).

In another particular embodiment a compound of Formula (V),

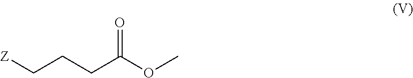

is used for the preparation of a compound of Formula (I).

In another particular embodiment a compound of Formula (VI),

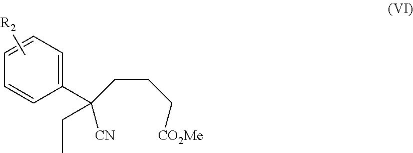

is used for the preparation of a compound of Formula (I).

In another particular embodiment a compound of Formula (VII),

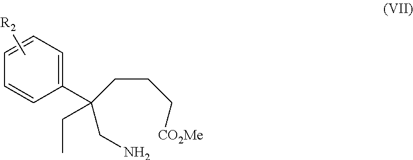

is used for the preparation of a compound of Formula (I).

In another particular embodiment a compound of Formula (VIII),

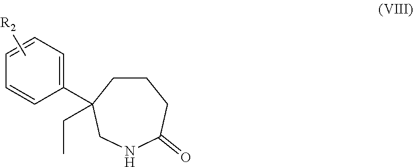

is used for the preparation of a compound of Formula (I).

In another particular embodiment a compound of Formula (IX),

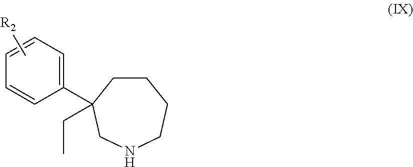

is used for the preparation of a compound of Formula (I).

In another particular embodiment a compound of Formula (Xa),

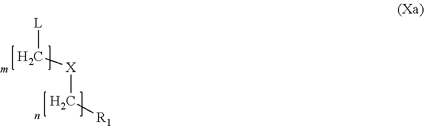

is used for the preparation of a compound of Formula (I).

In another particular embodiment a compound of Formula (Xa′),

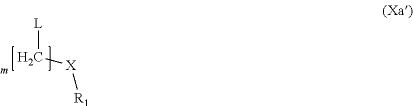

is used for the preparation of a compound of Formula (I).

In another particular embodiment a compound of Formula (Xb),

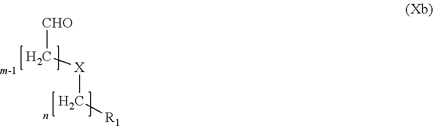

is used for the preparation of a compound of Formula (I).

In another particular embodiment a compound of Formula (Xb′),

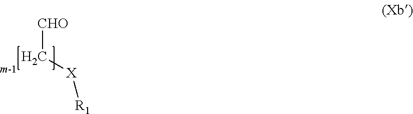

is used for the preparation of a compound of Formula (I).

In another particular embodiment a compound of Formula (Xc),

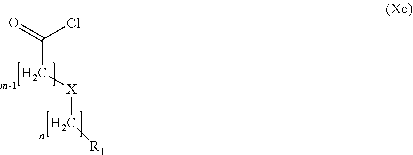

is used for the preparation of a compound of Formula (I).

In another particular embodiment a compound of Formula (Xc′),

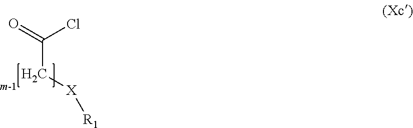

is used for the preparation of a compound of Formula (I).

In another particular embodiment a compound of Formula (XI),

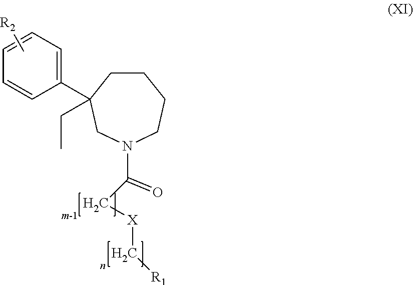

is used for the preparation of a compound of Formula (I).

In another particular embodiment a compound of Formula (XI′),

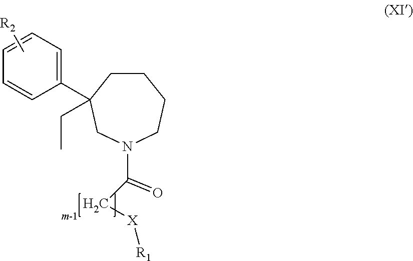

is used for the preparation of a compound of Formula (I).

The obtained reaction products may, if desired, be purified by conventional methods, such as crystallisation and chromatography. Where the above described processes for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

One preferred pharmaceutically acceptable form of a compound of the invention is the crystalline form, including such form in pharmaceutical composition. In the case of salts and also solvates of the compounds of the invention the additional ionic and solvent moieties must also be non-toxic. The compounds of the invention may present different polymorphic forms, it is intended that the invention encompasses all such forms.

Another aspect of the invention refers to a pharmaceutical composition which comprises a compound according to the invention as described above according to general formula I or a pharmaceutically acceptable salt or stereoisomer thereof, and a pharmaceutically acceptable carrier, adjuvant or vehicle. The present invention thus provides pharmaceutical compositions comprising a compound of this invention, or a pharmaceutically acceptable salt or stereoisomers thereof together with a pharmaceutically acceptable carrier, adjuvant, or vehicle, for administration to a patient

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatine, sorbitol, tragacanth, or polyvinylpyrrolidone; fillers, for example lactose, sugar, maize starch, calcium phosphate, sorbitol or glycine; tableting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to the use of a compound of the invention or a pharmaceutically acceptable salt or isomer thereof in the manufacture of a medicament.

Another aspect of the invention refers to a compound of the invention according as described above according to general formula I, or a pharmaceutically acceptable salt or isomer thereof, for use as a medicament for the treatment of pain. Preferably the pain is medium to severe pain, visceral pain, chronic pain, cancer pain, migraine, inflammatory pain, acute pain or neuropathic pain, allodynia or hyperalgesia. This may include mechanical allodynia or thermal hyperalgesia.

Another aspect of the invention refers to the use of a compound of the invention in the manufacture of a medicament for the treatment or prophylaxis of pain.

In a preferred embodiment the pain is selected from medium to severe pain, visceral pain, chronic pain, cancer pain, migraine, inflammatory pain, acute pain or neuropathic pain, allodynia or hyperalgesia, also preferably including mechanical allodynia or thermal hyperalgesia.

Another aspect of this invention relates to a method of treating or preventing pain which method comprises administering to a patient in need of such a treatment a therapeutically effective amount of a compound as above defined or a pharmaceutical composition thereof. Among the pain syndromes that can be treated are medium to severe pain, visceral pain, chronic pain, cancer pain, migraine, inflammatory pain, acute pain or neuropathic pain, allodynia or hyperalgesia, whereas this could also include mechanical allodynia or thermal hyperalgesia.

The present invention is illustrated below with the aid of examples. These illustrations are given solely by way of example and do not limit the general spirit of the present invention.

### EXAMPLES

### General Experimental Part (Methods and Equipment of the Synthesis and Analysis

A process is described in Scheme 1 or Scheme 1′ for the preparation of compounds of general formula I, wherein R₁, R₂, m, n and X have the meanings defined above, where, L is a leaving group such as halogen, mesylate, tosylate or triflate and Z is chloro, bromo or iodine.

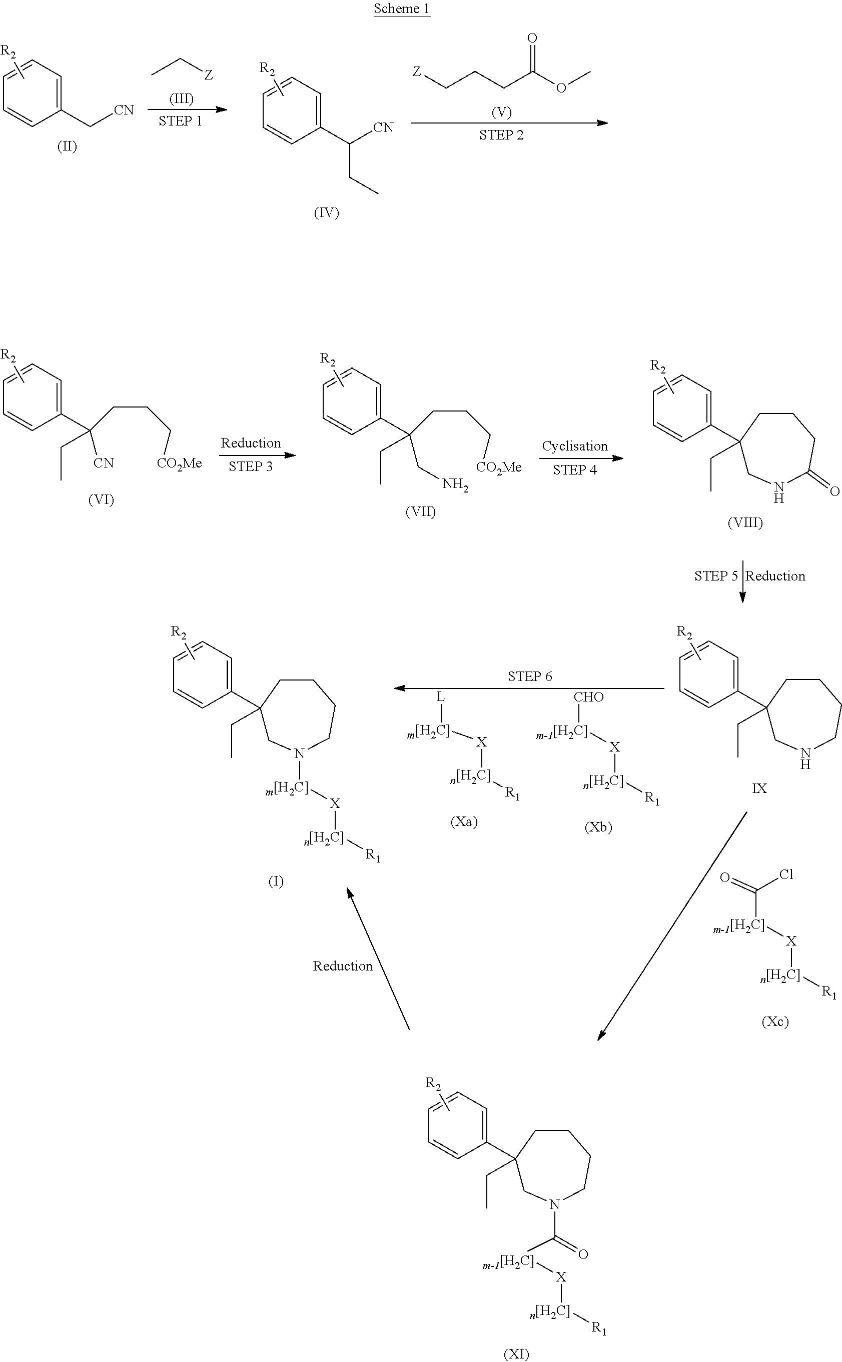

This process is carried out as described below:

Step 1: The compounds of formula IV are prepared by treating compounds of formula II with an alkylating reagent of formula III. This reaction may be carried out in a suitable solvent, such as DMF, in the presence of an inorganic base such as sodium hydride, at a suitable temperature comprised between room temperature and the reflux temperature, preferably at room temperature.
Step 2: A second alkylation is carried out on compounds of formula IV with alkylating reagents of formula V to render compounds of formula VI. This reaction can be performed under the conditions described in Step 1.
Step 3: The reduction of the nitrile group in a compound of formula VI renders a compound of formula VII. This reduction can be effected with hydrogen at a pressure comprised between 1 and 10 bars, in a suitable solvent such as methanol or ethanol, in the presence of palladium, at a suitable temperature comprised between room temperature and the reflux temperature, preferably at room temperature.
Step 4: The intramolecular cyclization reaction of a compound of formula VII to give a compound of formula VIII is carried out in the presence of a suitable solvent, such as xylene at a suitable temperature comprised between room temperature and the reflux temperature, preferably at the reflux temperature.
Step 5: Compounds of general formula IX can be prepared by reduction of lactam compounds of formula VIII with a suitable reagent such as LiAlH₄, in a suitable solvent such as THF, at a suitable temperature comprised between room temperature and the reflux temperature, preferably at room temperature.
Step 6: From compounds of formula IX, compounds of general formula I can be prepared by reaction with suitable reagents, such as those of formula Villa-b, using different conditions depending on the reagent nature. Thus:

The alkylation reaction with a compound of formula Xa is carried out in a suitable solvent, such as acetonitrile, dichloromethane, 1,4-dioxane, ethanol or dimethylformamide, preferably in acetonitrile, in the presence of an inorganic base such as K₂CO₃ or Cs₂CO₃, or an organic base such as triethylamine or N-ethyldiisopropylamine, preferably N-ethyldiisopropylamine, at a suitable temperature comprised between room temperature and the reflux temperature, preferably heating, or alternatively, this reaction can be carried out in a microwave reactor. Additionally, an activating agent such as NaI or KI can be used.

The reductive amination with a compound of formula Xb, is carried out in the presence of a reductive reagent, such as sodium triacetoxyborohydride, in an aprotic solvent, preferably tetrahydrofuran or dichloroethane, at a suitable temperature comprised between room temperature and the reflux temperature, or alternatively, this reaction can be carried out in a microwave reactor.

Alternatively, the transformation of a compound of formula IX to a compound of formula I, can be effected in a two step procedure, involving acylation of IX with an acid chloride of formula Xc to give a compound of formula XI, which is then reduced. The acylation reaction can be carried out using triethylamine in a suitable solvent such as dichloromethane at a suitable temperature, preferably room temperature. The reduction reaction can be effected with a reducing agent such as LiAlH₄, in a suitable solvent such as tetrahydrofuran, at a suitable temperature comprised between 0° C. and room temperature, preferably at room temperature.

The process described by Steps 1 to 6 represent the general route for the preparation of compounds of formula I. Additionally, the functional groups present in any of the positions can be interconverted using reactions known to those skilled in the art. As a matter of example a compound of formula I can be converted into another compound of formula I by transforming a hydroxyl group in position R₂ into an amino group. This transformation can involve conversion of the hydroxyl group into a triflate, reaction of this triflate with diphenylmethanimine under metal catalysed conditions and hydrolysis of the protecting group under acidic conditions to provide a free amino functionality. A compound of formula I were R₂ is amino can be converted into an alkyl sulfonylamino group by reaction with a suitable alkyl sulfonyl chloride in the presence of a base such as pyridine.

Compounds of formula II, III, V, and Xa-c where R₁, m, n, L, X and Z have the meanings as defined above, are commercially available or can be prepared by conventional methods described in the bibliography.

Preferably the methods of Scheme 1 are methods of Scheme 1′ wherein n is 0:

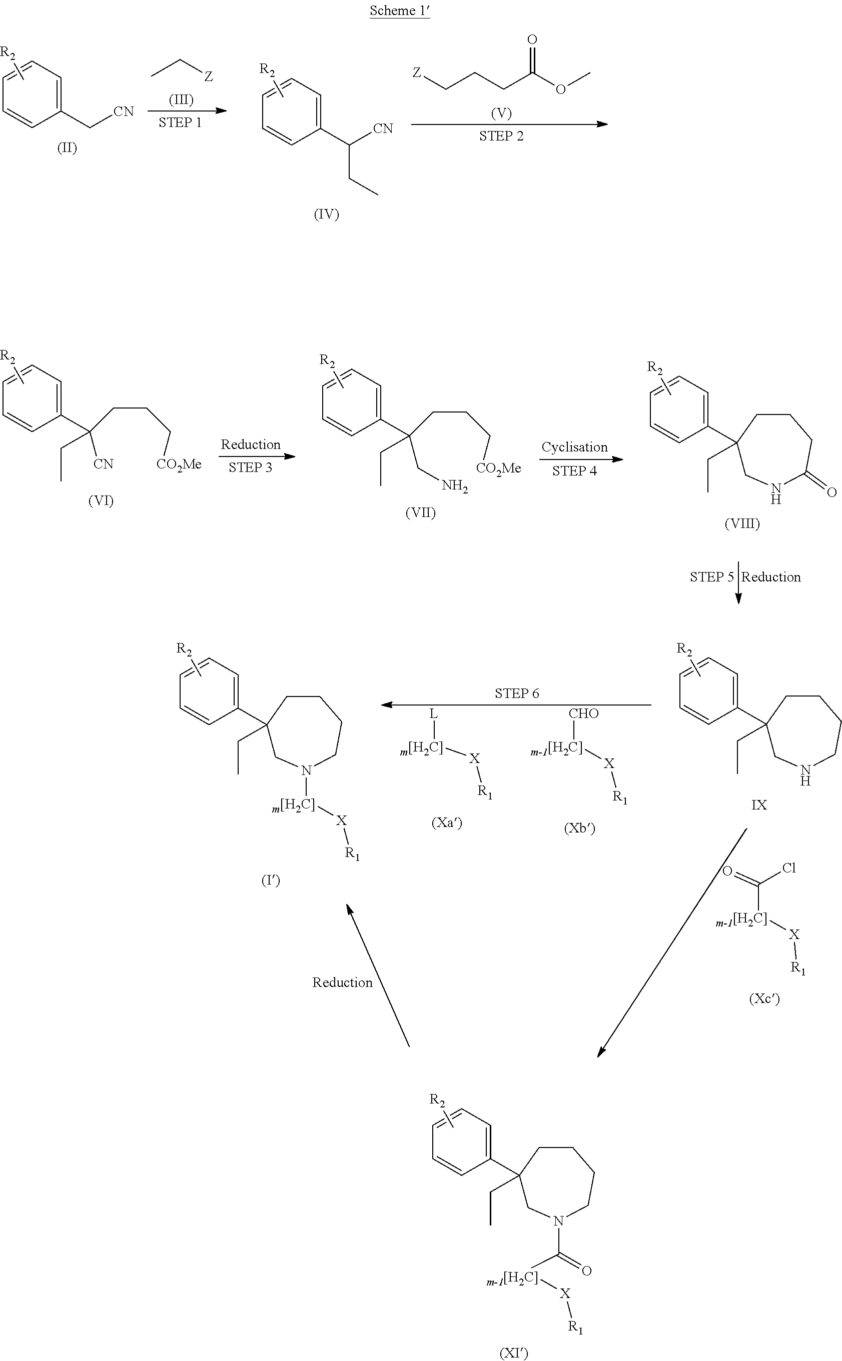

### Examples

### Intermediates and Examples

The following abbreviations are used in the examples:

### ACN: Acetonitrile

AcOEt: Ethyl acetate

### Anh: Anhydrous

### Conc: Concentrated

### CH: Cyclohexane

### DCM: Dichloromethane

### DIPEA: N,N-Diisopropytethylamine

### EtOH: Ethanol

Et₂O: Diethyl ether

### Ex: Example

### h: Hour/s

HPLC: High-performance liquid chromatography
H₂O: water

### MeOH: Methanol

MS: Mass spectrometry

### Min: Minutes

### Quant: Quantitative

### Ret: Retention

rt: Room temperature

### Sat: Saturated

### TEA: Et₃N, Triethylamine

### THF: Tetrahydrofuran

### Wt: Weight

The following methods were used to obtain the HPLC-MS data;

A: Column Acquity UPLC BEH C18 2.1×50 mm, 1.7 μm; flow rate 0.61 mL/min; A: NH₄HCO₃ 10 mM; B: ACN; Gradient: 0.3 min in 98% A, 98% A to 5% A in 2.52 min, 1.02 min in 5% A, 5% A to 98% A in 0.34 min, 0.57 min in 98% A.

B Column SunFire C18 2.1×100 mm, 3.5 μm; flow rate 0.3 mU/min; A*ACN:MeOH (1:1), B: Water, C: Ammonium Acetate 100 mM pH 7 Gradient: 3 min 10:85:5 A:B:C, 10:85:5 AB:C to 95:0:5 A:B:C in 10 min. 10 min 95:0:5 A:B:C C: Column SunFire C18 2.1×100 mm, 3.5 μm; flow rate 0.3 mU/min; A: ACN:MeOH (1:1), B: Water, C: Ammonium Acetate 100 mM pH 9 Gradient: 3 min 10:85:5 A:B:C, 10:85:5 A:B:C to 95:0:5 A:B:C in 10 min, 10 min 95:0:5 A:B:C.

D: Column Luna C18 (2) 5 μm, 2.0×50 mm; flow rate: 0.30 mU/min; A: ACN:MeOH (1:1); B: Water; C: 100 mM Ammonium acetate pH 7; gradient A:B:C: 3 min in 10:85:5+from 10:85:5 to 95:0:5 in 6 min+6 min in 95:0:5].

### Intermediate 1. 2-(3-Methoxyphenyl)butanenitrile

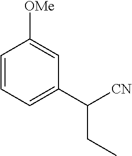

Over a solution of 2-(3-methoxyphenyl)acetonitrile (5.0 g, 33.97 mmol) in DMF (50 mL) cooled at 0° C., NaH (60% wt, 1.63 g, 40.76 mmol) was added portionwise and the mixture stirred at rt for 30 min. Then it was cooled back to 0° C., and bromoethane (3.04 mL, 40.76 mmol) was added. The reaction mixture was allowed to reach rt and stirred until full conversion was achieved. Then mixture was poured into H₂O (100 mL) and the aqueous phase was extracted with AcOEt. The combined organic layers were dried over anh Na₂SO₄, filtered and concentrated to dryness. The crude thus obtained was purified by column chromatography on silica (0-5% AcOEt/Hexane) to give the title compound as colourless oil (3.17 g, yield 53%).

RMN—¹H (CDCl₃, 250 MHz, δ): 7.29 (m, 1H, ArH); 6.95-6.81 (m, 3H, ArH); 3.82 (s, 3H, OCH₃); 3.71 (t, J=7.1 Hz, 1H, CH); 1.94 (q, J=7.4 Hz, 2H, CH₂); 1.07 (t, J=7.4 Hz, 3H, CH₃).

### Intermediate 2. Methyl 5-cyano-5-(3-methoxyphenyl)heptanoate

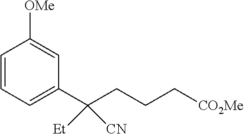 [SMILES: CCC(C#N)(CCCC(=O)OC)c1cccc(OC)c1]

To a solution of 2-(3-methoxyphenyl)butanenitrile (intermediate 1, 1.0 g, 5.70 mmol) in DMF (50 mL) cooled at −30° C., NaH (60% wt, 320 mg, 7.98 mmol) was added portionwise and the mixture was stirred for 10 min. Methyl 4-iodobutanoate (1.82 g, 7.98 mmol) was subsequently added and the mixture was allowed to reach rt. The mixture was stirred at this temperature for 14 h and was poured into H₂O (75 mL). The aqueous phase was extracted with AcOEt and the combined organic phases were dried over anh. Na₂SO₄, filtered and concentrated to dryness. The crude thus obtained was purified by column chromatography on silica (5-10% AcOEt/Hexane) to give the title compound as colourless oil (2.03 g, yield 78%).

RMN—¹H (CDCl₃, 250 MHz, δ): 7.3 (t, J=7.9 Hz, 1H, ArH); 7.0-6.9 (m, 2H, ArH); 8.8 (dd, J=8.2, 2.4 Hz, 1H, ArH); 3.83 (s, 3H, OCH₃); 3.63 (s, 3H, CO₂CH₃); 2.29 (dt, J=7.4, 2.2 Hz, 2H, CH₂); 2.1-1.2 (m, 6H, CH₂); 0.9 (t, J=7.1 Hz, 3H, CH₃).

### Intermediate 3. Methyl 5-(aminomethyl)-5-(3-methoxyphenyl)heptanoate

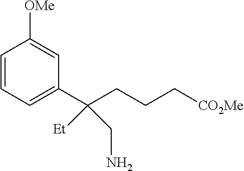 [SMILES: CCC(CN)(CCCC(=O)OC)c1cccc(OC)c1]

A mixture of methyl 5-cyano-5-(3-methoxyphenyl)heptanoate (intermediate 2, 1.25 g, 4.53 mmol) and palladium (927 mg, 0.453 mmol, 10% wt on charcoal wet) in MeOH (20 mL) and H₂SO₄ cc (1.2 mL) was stirred at rt under 1 bar of H₂ overnight. Then, the solids were filtered off over a pad of celite and the solvent was evaporated to dryness. The residue was dissolved in DCM (30 mL) and water (20 mL), made alkaline with aqueous NaOH (36%), and extracted twice with DCM. The combined organic phases were dried over anh. Na₂SO₄, filtered and concentrated to give the title compound as pale brown oil (1.02 g, 81% yield)

RMN—¹H (CDCl₃, 250 MHz, δ): 7.34-7.18 (m, 3H, ArH+NH₂); 6.94-6.80 (m, 2H, ArH); 6.74 (dd, J=7.4, 2.2 Hz, 1H, ArH); 3.81 (s, 3H, OCH₃); 3.61 (s, 3H, CO₂CH₃); 2.89 (s, 2H, CH₂); 2.29 (t, J=7.1 Hz, 2H, CH₂); 1.80-1.18 (m, 6H, CH₂); 0.74 (t, J=7.4 Hz, 3H, CH₃).

HPLC-MS (Method A): Ret, 1.70 min; ESI⁺-MS m/z, 294.0 (M+H).

### Intermediate 4. 6-Ethyl-6-(3-methoxyphenyl)azepan-2-one

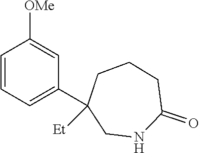 [SMILES: CCC1(c2cccc(OC)c2)CCCC(=O)NC1]

A solution of methyl 5-(aminomethyl)-5-(3-methoxyphenyl)heptanoate (Intermediate 3, 1.92 g, 6.87 mmol) in xylene (13 mL) was refluxed for 4.5 h. The reaction mixture was stirred until full conversion was achieved and then it was concentrated to dryness. The crude product thus obtained was purified by column chromatography on silica (3% MeOH/DCM) to give the title compound as dense yellow oil (1.38 g, 81% yield).

RMN—¹H (CDCl₃, 250 MHz, δ): 7.27 (m, 1H, ArH); 6.90-6.68 (m, 3H, ArH); 5.93 (m, 1H, NH); 3.81 (s, 3H, OCH₃); 3.47 (m, 2H, CH₂); 2.59-2.17 (m, 3H, CH₂); 1.78 (m, 3H, CH₂); 1.61 (c, J=7.4 Hz, 2H, CH₂); 0.61 (t, J=7.4 Hz, 3H, CH₃).

HPLC-MS (Method A): Ret, 1.59 min; ESI⁺-MS m/z, 248.2 (M+H).

### Intermediate 5. 3-Ethyl-3-(3-methoxyphenyl)azepane

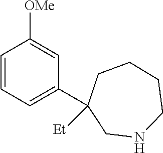 [SMILES: CCC1(c2cccc(OC)c2)CCCCNC1]

A solution of 6-ethyl-6-(3-methoxyphenyl)azepan-2-one (intermediate 4, 1.38 g, 5.57 mmol) in THF (25 mL) was added to a stirred solution of warm LiAlH₄ (1.0 M in THF, 12 mL, 11.69 mmol). The mixture was refluxed for 2 h until full conversion was achieved and then it was cooled at −20° C. Aqueous NaOH solution (15%, 0.45 mL) and water (2 mL) were added and the resulting mixture was allowed to reach rt, stirred at this temperature for 15 min and filtered. The precipitate was washed with EtOAc and the resulting filtrate was washed with water (20 mL). The combined organic phases were dried over anh. Na₂SO₄, filtered and concentrated to dryness to give the title compound (1.12 g, 86% yield).

RMN—¹H (CDCl₃, 250 MHz, δ): 7.25 (m, 1H, ArH); 6.95-6.83 (m, 2H, ArH); 6.73 (ddd, J=8.2, 2.7 y 0.8 Hz, 1H, ArH); 3.81 (s, 3H, OCH₃); 3.23 (d, J=14.2 Hz, 1H, CH₂); 2.86 (d, J=14.2 Hz, 1H, CH₂); 2.77 (m, 2H, CH₂); 2.23 (m, 1H, NH); 1.80-1.40 (m, 8H, CH₂); 0.59 (t. J=7.4 Hz, 3H, CH₃).

HPLC retention time (method A): 1.63 min; MS: 234.2 (M+H).

### Example 1. 3-Ethyl-3-(3-methoxyphenyl)-1-(2-(4-methylpiperazin-1-yl)ethyl)azepane

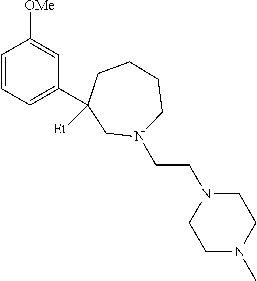 [SMILES: CCC1(c2cccc(OC)c2)CCCCN(CCN2CCN(C)CC2)C1]

### a) 2-Chloro-1-(3-ethyl-3-(3-methoxyphenyl)azepan-1-yl)ethanone

2-Chloroacetyl chloride (435.6 mg, 3.86 mmol) was added to a solution of 3-ethyl-3-(3-methoxyphenyl)azepane (intermediate 5, 300 mg, 1.29 mmol) and TEA (448 μL, 3.21 mmol) in DCM (15 mL) under nitrogen atmosphere and the reaction mixture was stirred at rt for 1.5 h. Then, the mixture was washed twice with water and the organic layer was dried with anh. Na₂SO₄, filtered and concentrated to dryness to give the title compound as oil (360 mg, yield 90%).

### b) 1-(3-Ethyl-3-(3-methoxyphenyl)azepan-1-yl)-2-(4-methylpiperazin-1-yl)ethanone

1-Methylpiperazine (107 mg, 1.06 mmol) was added to a solution of 2-chloro-1-(3-ethyl-3-(3-methoxyphenyl)azepan-1-yl)ethanone (obtained in step a, 220 mg, 0.71 mmol) and K₂CO₃ (491 mg, 3.55 mmol) in ACN (10 mL) cooled to 0° C. The reaction mixture was stirred at rt for 3 h, and refluxed at 80° C. for 3 h more. The reaction mixture was poured into H₂O (20 mL) and extracted with AcOEt. The combined organic layers were dried over anh Na₂SO₄, filtered and concentrated to dryness to give the title compound as yellow oil (250 mg, 95% yield).

### c) Title Compound

A solution of 1-(3-ethyl-3-(3-methoxyphenyl)azepan-1-yl)-2-(4-methylpiperazin-1-yl)ethanone (obtained in step b, 113 mg, 0.30 mmol) in THF (5 mL) was added to a stirred solution of LiAlH₄ (1.0 M in THF, 0.6 mL, 0.63 mmol). The mixture was stirred for 19 h at rt and then it was cooled at −20° C. Then, NaOH aqueous solution and water were added. The resulting mixture was allowed to reach rt, stirred at this temperature for 15 min and filtered. The precipitate was washed with AcOEt and the resulting filtrate was washed with water. The obtained organic phase was dried over anh. Na₂SO₄, filtered and concentrated to dryness to give the title compound (55 mg, 51% yield).

HPLC-MS (Method C): Ret, 17.67 min; ESI⁺-MS rWz 360 (M+H).

This method was used for the preparation of examples 2-5 using suitable starting materials:

| | | | | Ret | MS |
|---|---|---|---|---|---|
| EX | Structure | Chemical name | Method | (min) | (M + H) |
| 2 | 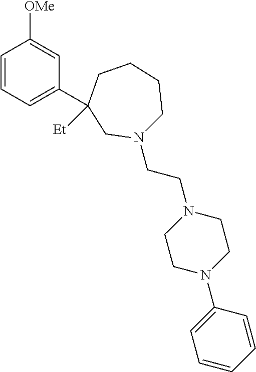 [SMILES: CCC1(c2cccc(OC)c2)CCCCN(CCN2CCN(c3ccccc3)CC2)C1] | 3-ethyl-3-(3- methoxyphenyl)-1-(2- (4-phenylpiperazin-1- yl)ethyl)azepane | C | 21.03 | 421.62 |
| 3 | 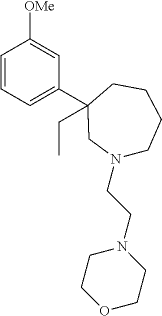 [SMILES: CCC1(c2cccc(OC)c2)CCCCN(CCN2CCOCC2)C1] | 4-(2-(3-Ethyl-3-(3- methoxyphenyl)azepan- 1-yl)ethyl)morpholine | A | 2.20 | 347.3 |
| 4 | 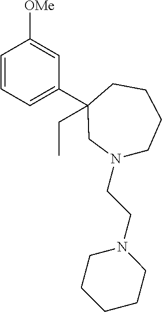 [SMILES: CCC1(c2cccc(OC)c2)CCCCN(CCN2CCCCC2)C1] | 3-ethyl-3-(3- methoxyphenyl)-1-(2- (piperidin-1- yl)ethyl)azepane | A | 2.34 | 345.5 |
| 5 | 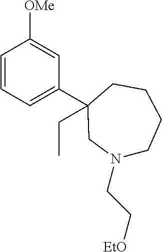 [SMILES: CCOCCN1CCCCC(CC)(c2cccc(OC)c2)C1] | 1-(2-ethyoxyethyl)- 3-ethyl-3-(3- methoxyphenyl)azepane | A | 2.64 | 306.2 |

### Example 6. 3-(3-Ethyl-1-(2-(4-methylpiperazin-1-yl)ethyl)azepan-3-yl)phenol

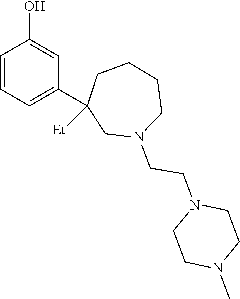 [SMILES: CCC1(c2cccc(O)c2)CCCCN(CCN2CCN(C)CC2)C1]

A solution of tribromoborane (1 M in DCM, 0.31 mmol, 0.31 mL) was added to a solution of 3-ethyl-3-(3-methoxyphenyl)-1-(2-(4-methylpiperazin-1-yl)ethyl)azepane (Example 1, 37 mg, 0.10 mmol) in DCM (5 mL) cooled to −40° C. The mixture was allowed to reach rt and stirred for 4.5 h. The reaction mixture was cooled to 0° C. and a saturated aqueous solution of sodium bicarbonate was added. The organic phase was washed with water, dried over anh Na₂SO₄, and filtered. The solvent was removed and the crude product thus obtained was purified by flash chromatography on silica, gradient Cl₂CH₂/MeOH (0 to 20%) to give the title compound (14 mg, 39% yield).

HPLC-MS (Method B): Ret, 19.49 min; ESI⁺-MS m/z 346 (M+H).

This method was used for the preparation of example 7-10 using example 2-5 as starting material:

| | | | | Ret | MS |
|---|---|---|---|---|---|
| EX | Structure | Chemical name | Method | (min) | (M + H) |
| 7 | 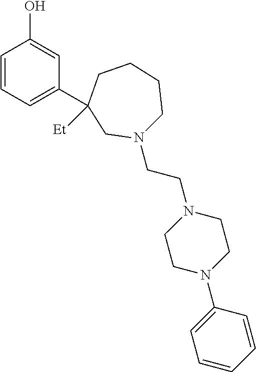 [SMILES: CCC1(c2cccc(O)c2)CCCCN(CCN2CCN(c3ccccc3)CC2)C1] | 3-ethyl-1-(2-(4- phenylpiperazin)-1- yl)ethyl)azepan-3- yl)phenol | B | 18.35 | 408 |
| 8 | 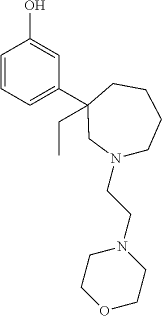 [SMILES: CCC1(c2cccc(O)c2)CCCCN(CCN2CCOCC2)C1] | 3-(3-ethyl-1-(2- morpholinoethyl)azepan- 3-yl)phenol | A | 1.74 | 333 |
| 9 | 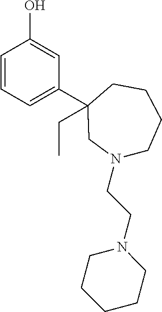 [SMILES: CCC1(c2cccc(O)c2)CCCCN(CCN2CCCCC2)C1] | 3-ethyl-1-(2- (piperidin-1- yl)ethyl)azepan-3- yl)phenol | A | 1.92 | 331.3 |
| 10 | 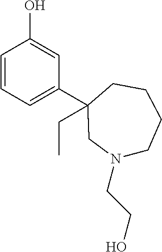 [SMILES: CCC1(c2cccc(O)c2)CCCCN(CCO)C1] | 3-(3-ethyl-1-(2- hydroxyethyl)azepan- 3-yl)phenol | A | 1.59 | 264.2 |

### Example 11. 3-(1-(2-Ethoxyethyl)-3-ethylazepan-3-yl)phenol

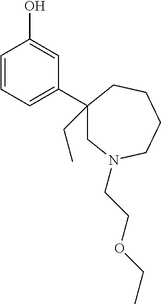 [SMILES: CCOCCN1CCCCC(CC)(c2cccc(O)c2)C1]

### a) 3-(3-Ethylazepan-3-yl)phenol

The title compound was obtained following the procedure described in example 6, and using 3-ethyl-3-(3-methoxyphenyl)azepane (intermediate 5) as starting material.

HPLC-MS (Method C): Ret, 1.26 min; ESI⁺-MS m/z, 220.2 (M+H).

### b) Title Compound

To a solution of 3-(3-ethylazepan-3-yl)phenol (step a, 0.42 g, 1.92 mmol) in ACN (20 mL), N-ethyldiisipropylamine (0.67 mL, 3.830 mmol) and 1-bromo-2-ethoxyethane (0.24 mL, 2.11 mmol) were added and the reaction mixture was heated at 65° C. overnight. The reaction mixture was cooled and partitioned between 5% aqueous KHCO₃ solution and AcOEt. The layers were separated and the organic layer was dried over anh Na₂SO₄, filtered and concentrated to dryness. The crude product thus obtained was purified by flash chromatography on silica gel, gradient DCM/MeOH (0 to 10% MeOH) to give the title compound (0.15 g, 28% yield).

HPLC-MS (Method A): Ret, 2.12 min; ESI⁺-MS m/z, 292.4 (M+H).

This method was used for the preparation of Examples 12-19:

| | | | | Ret | MS |
|---|---|---|---|---|---|
| EX | Structure | Chemical name | Method | (min) | (M + H) |
| 12 | 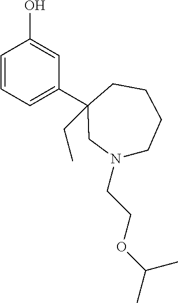 [SMILES: CCC1(c2cccc(O)c2)CCCCN(CCOC(C)C)C1] | 3-(3-ethyl-1-(2- isopropoxyethyl))azepan- 3-yl)phenol | A | 2.24 | 306 |
| 13 | 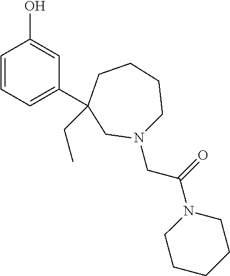 [SMILES: CCC1(c2cccc(O)c2)CCCCN(CC(=O)N2CCCCC2)C1] | 2-(3-ethyl-3-(3- hydroxyphenyl)azepan- 1-yl)-1-(piperidin-1- yl)ethanone | A | 2.12 | 345 |
| 14 | 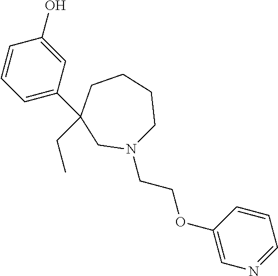 [SMILES: CCC1(c2cccc(O)c2)CCCCN(CCOc2cccnc2)C1] | 3-(3-ethyl-1-(2- (pyridin-3- yloxy)ethyl)azepan-3- yl)phenol | A | 2.06 | 341 |
| 15 | 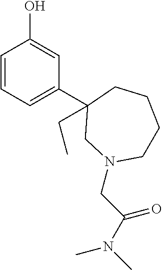 [SMILES: CCC1(c2cccc(O)c2)CCCCN(CC(=O)N(C)C)C1] | 2-(3-ethyl-3-(3- hydroxyphenyl)azepan- 1-yl)-N,N- dimethylacetamide | A | 1.87 | 338 |
| 16 | 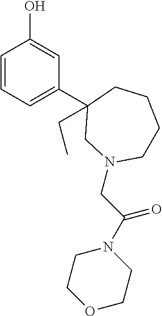 [SMILES: CCC1(c2cccc(O)c2)CCCCN(CC(=O)N2CCOCC2)C1] | 2-(3-ethyl-3-(3- hydroxyphenyl)azepan- 1-yl)-1- morpholinoethanone | A | 1.79 | 347 |
| 17 | 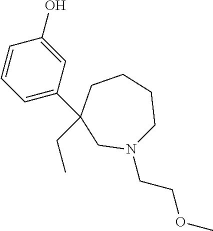 [SMILES: CCC1(c2cccc(O)c2)CCCCN(CCOC)C1] | 3-(3-ethyl-1-(2- methoxyethyl)azepan- 3-yl)phenol | A | 1.96 | 278 |
| 18 | 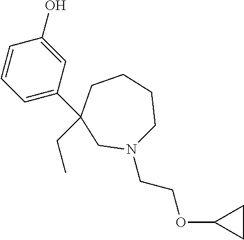 [SMILES: CCC1(c2cccc(O)c2)CCCCN(CCOC2CC2)C1] | 3-(1-(2- cyclopropoxyethyl)-3- ethylazepan-3- yl)phenol | A | 2.26 | 304 |
| 19 | 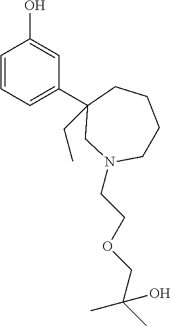 [SMILES: CCC1(c2cccc(O)c2)CCCCN(CCOCC(C)(C)O)C1] | 3-(3-ethyl-1-(2-(2- hydroxy-2- methylpropoxy)ethyl) azepan-3-yl)phenol | A | 1.83 | 336 |

### Examples 20 and 21. (S) and (R)-3-(1-(2-ethoxyethyl)-3-ethylazepan-3-yl)phenol

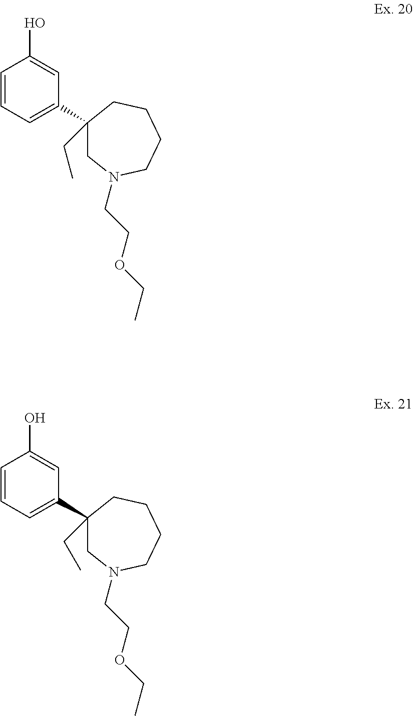 [SMILES: CCOCCN1CCCC[C@@](CC)(c2cccc(O)c2)C1 ; CCOCCN1CCCC[C@](CC)(c2cccc(O)c2)C1]

The enantiomers of example 11 were separated by preparative HPLC using Chiralcel OJ Column, n-Heptane/(EtOH+0.33% DEA) 90/10 v/v, it to give the title compounds.

HPLC-MS (Method A): Ret, 2.11 min; ESI⁺-MS m/z, 292.2 (M+H).

### Example 22. 3-[1-(2-Ethoxyethyl)-3-ethylazepan-3-yl]aniline

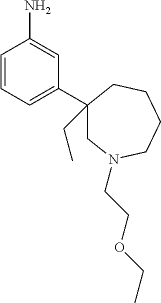 [SMILES: CCOCCN1CCCCC(CC)(c2cccc(N)c2)C1]

### a) 3-[1-(2-Ethoxyethyl)-3-ethylazepan-3-yl]phenyl trifluoromethanesulfonate

Trifluoromethanesulfonic anhydride (0.32 mL, 1.94 mmol) and DIPEA (0.41 mL, 2.42 mmol) were added to a −50° C. cooled solution of 3-[1-(2-ethoxyethyl)-3-ethylazepan-3-yl]phenol (Example 11, 0.47 g, 1.61 mmol) in DCM (16 mL). After 15 min the reaction mixture was diluted with DCM (30 mL) and allowed to reach rt. The resulting solution was washed with water (30 mL) and the organic layer was dried over anh Na₂SO₄, filtered and concentrated. The crude residue was purified by flash chromatography on SiO₂ (30% EtOAc/hexanes), affording the title compound as pale yellow oil (0.31 g, 46% yield).

HPLC-MS (Method 0): Ret, 12.4 min; ESI+-MS m/z, 424 (M+H).

### b) N-{3-[1-(2-Ethoxyethyl)-3-ethylazepan-3-yl]phenyl}-1,1-diphenylmethanimine

Pd₂(dba)₃ (33 mg, 0.04 mmol) was added to a degassed mixture of John-Phos (33 mg, 0.11 mmol), K₃PO₄ (0.46 g, 2.19 mmol), 1,1-diphenylmethanimine (0.16 mL, 0.95 mmol) and the compound obtained in step a (0.31 g, 0.73 mmol) in DME (12 mL) and the resulting suspension was heated at 70° C. for 24 h. The reaction mixture was allowed to cool down to rt and volatiles were removed in the presence of SiO₂. The residue was purified by medium pressure flash chromatography (Combiflash, 0 to 100% EtOAc/hexanes) to give the title compound as pale orange oil (0.185 g, 56% yield).

HPLC-MS (Method D): Ret, 12.68 min; ESI+-MS m/z: 455 (M+1).

### c) Title Compound

HCl (6M aqueous solution, 1.33 mL, 8.00 mmol) was added dropwise to solution of the compound obtained in step b (0.18 g, 0.40 mmol) in THF (6 mL). After 6 h the reaction mixture was concentrated and the residue was diluted with DCM (20 mL), basified with NaOH (10% aqueous solution, 6 mL) and washed with water (10 mL). The organic layer was dried over anh Na₂SO₄, filtered and concentrated. The crude residue was purified by flash chromatography on SiO₂ (5%-10% MeOH/DCM) affording the title compound as brown oil (67% yield).

HPLC-MS (Method D): Ret, 9.50 min; ESI+-MS m/z: 291 (M+1).

### Example 23. N-{3-[1-(2-Ethoxyethyl)-3-ethylazepan-3-yl]phenyl}methanesulfonamide

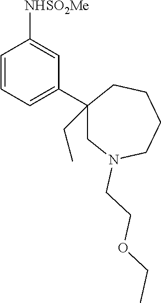

Methanesulfonyl chloride (23 μL, 0.29 mmol) was added to a 0° C. cooled solution of the compound obtained in example 22 (70 mg, 0.24 mmol) and pyridine (39 μL, 0.48 mmol) in DCM (10 mL). The reaction mixture was allowed to reach rt and stirred for 20 h. Volatiles were removed in the presence of SiO₂ and the residue was purified by flash chromatography on SiO₂ (5 to 10% MeOH/DCM) to afford the title compound as a sticky yellow solid (75 mg, 83% yield).

HPLC-MS (Method B): Ret, 17.79 min: ESI+-MS m/z: 369 (M+1).

### Example 24. N-{3-[1-(2-Ethoxyethyl)-3-ethylazepan-3-yl]phenyl}propane-2-sulfonamide

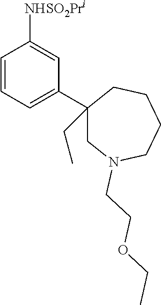

Starting from the compound obtained in example 22 and employing the same method described in example 23, but using propane-2-sulfonyl chloride as sulfonylation reagent instead of methanesulfonyl chloride, the title compound was obtained in 19% yield.

HPLC-MS (Method B): Ret, 19.55 min; ESI+-MS m/z: 397 (M+1).

Table of Examples with Binding to the μ-Opioid Receptor and the σ₁-Receptor:

### Biological Activity

### Pharmacological Study

### Human σ₁ Receptor Radioligand Assay

To investigate binding properties of test compounds to human or receptor, transfected HEK-293 membranes and [³H](+)-pentazocine (Perkin Elmer, NET-1056), as the radioligand, were used. The assay was carried out with 7 μg of membrane suspension, 5 nM of [³H](+)-pentazocine in either absence or presence of either buffer or 10 μM Haloperidol for total and non-specific binding, respectively. Binding buffer contained Tris-HCl 50 mM at pH 8. Plates were incubated at 37° C. for 120 minutes. After the incubation period, the reaction mix was then transferred to MultiScreen HTS, FC plates (Millipore), filtered and plates were washed 3 times with ice-cold 10 mM Tris-HCL (pH7.4). Filters were dried and counted at approximately 40% efficiency in a MicroBeta scintillation counter (Perkin-Elmer) using EcoScint liquid scintillation cocktail

### Human μ-Opioid Receptor Radioligand Assay

To investigate binding properties of test compounds to human μ-opioid receptor, transfected CHO-K1 cell membranes and [³H]-DAMGO (Perkin Elmer, ES-542-C), as the radioligand, were used. The assay was carried out with 20 μg of membrane suspension, 1 nM of [³H]-DAMGO in either absence or presence of either buffer or 10 μM Naloxone for total and non-specific binding, respectively. Binding buffer contained Tris-HCl 50 mM, MgCl2 5 mM at pH 7.4. Plates were incubated at 27° C. for 60 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

### Results:

As this invention is aimed at providing a compound or a chemically related series of compounds which act as dual ligands of the σ₁ receptor and the μ-opioid receptor it is a very preferred embodiment in which the compounds are selected which act as dual ligands of the σ₁ receptor and the μ-opioid receptor and especially compounds which have a binding expressed as K1 which is preferably <1000 nM for both receptors, more preferably <500 nM, even more preferably <100 nM.

The following scale has been adopted for representing the binding to the σ₁ receptor and the μ-opioid receptor expressed as K4:
+ Both Kᵢ-μ and Kᵢ-σ₁>=500 nM
++ One Kᵢ <500 nM while the other Kᵢ is >=500 nM
+++ Both Kᵢ-μ and Kᵢ-σ₁ <500 nM
++++ Both Kᵢ-μ and Kᵢ-σ₁ <100 nM

All compounds prepared in the present application exhibit binding to the or receptor and the μ-opioid receptor, in particular the following binding results are shown:

| | μ and |
|---|---|
| | σ₁ dual |
| EX | binding |
| 1 | + |
| 2 | +++ |
| 3 | + |
| 4 | + |
| 5 | + |
| 6 | ++ |
| 7 | ++ |
| 8 | ++ |
| 9 | +++ |
| 10 | ++ |
| 11 | ++++ |
| 12 | +++ |
| 13 | ++ |
| 14 | ++ |
| 15 | ++ |
| 16 | + |
| 17 | ++ |
| 18 | ++ |
| 19 | ++ |
| 20 | ++ |
| 21 | ++ |
| 22 | + |
| 23 | + |
| 24 | + |

1-14. (canceled)

15. A compound of Formula (I): 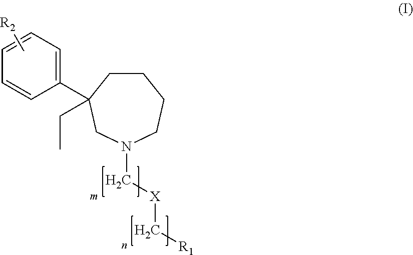
wherein
m is 1, 2 or 3;
n is 0, 1 or 2;
X is selected from the group consisting of —CH₂N(R_{1′})—, —C(O)N(R_{1′})— and —CH₂O—;
R₁ is selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, and substituted or unsubstituted heterocyclyl;
R_{1′} is selected from the group consisting of substituted or unsubstituted C₁₋₆ alkyl, substituted or unsubstituted C₂₋₆ alkenyl, substituted or unsubstituted C₂₋₆ alkynyl; alternatively, when X is —CH₂N(R_{1′})— or —C(O)N(R_{1′})—, R₁ and R_{1′} taken together with the connecting N—[CH₂]ₙ atoms may form a substituted or unsubstituted heterocyclyl having up to 6 ring members;
wherein the alkyl, alkenyl or alkynyl in R₁ or R_{1′}, if substituted, is substituted with one or more substituents selected from the group consisting of —OR₃, halogen, —CN, haloalkyl, haloalkoxy and —NR₃R_{3′″};
wherein
R₃ is selected from the group consisting of hydrogen, unsubstituted C₁₋₆ alkyl, unsubstituted C₂₋₆ alkenyl and unsubstituted C₂₋₆ alkynyl;
and wherein R_{3′″} is selected from the group consisting of hydrogen, unsubstituted C₁₋₆ alkyl, unsubstituted C₂₋₆ alkenyl, unsubstituted C₂₋₆ alkynyl and -Boc;
R₂ is selected from the group consisting of hydrogen, halogen, —R₄, —OR₄, —NO₂, —NR₄R_{4′″}, —NR₄C(O)R_{4′}, —NR₄S(O)₂R_{4′}, —S(O)₂NR₄R_{4′}, —NR₄C(O)NR_{4′}R_{4″}, —SR₄, —S(O)R₄, —S(O)₂R₄, —OS(O)₂R₄, —CN, haloalkyl, haloalkoxy, —C(O)OR₄, —C(O)NR₄R_{4′}, —OCH₂CH₂OH, —NR₄S(O)₂NR_{4′}R_{4″} and —C(CH₃)₂OR₄; wherein
R₄, R_{4′} and R_{4″} are independently selected from the group consisting of hydrogen, unsubstituted C₁₋₆ alkyl, unsubstituted C₂₋₆ alkenyl, and unsubstituted C₂₋₆ alkynyl;
R_{4′″} is selected from hydrogen, unsubstituted C₁₋₆ alkyl, unsubstituted C₂₋₆ alkenyl, unsubstituted C₂₋₆ alkynyl and -Boc;
optionally as a stereoisomer, including enantiomers and diastereomers, a racemate, or a mixture of at least two stereoisomers, including enantiomers and/or diastereomers, in any mixing ratio, or a corresponding salt thereof, or a corresponding solvate thereof.

16. The compound according to claim 15, wherein the compound of Formula (I) is a compound of formula (I′): 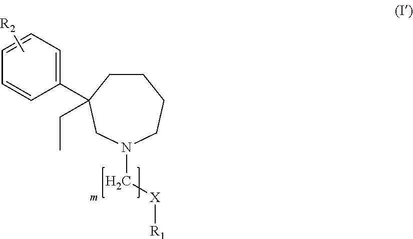
wherein
m is 1, 2 or 3;
X is selected from the group consisting of —CH₂N(R_{1′})—, —C(O)N(R_{1′})— and —CH₂O—;
R₁ is selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, and substituted or unsubstituted heterocyclyl;
R_{1′} is selected from the group consisting of substituted or unsubstituted C₁₋₆ alkyl, substituted or unsubstituted C₂₋₆ alkenyl, and substituted or unsubstituted C₂₋₆ alkynyl;
alternatively, when X is —CH₂N(R_{1′})— or —C(O)N(R_{1′})—, R₁ and R_{1′} taken together with the connecting N atom may form a substituted or unsubstituted heterocyclyl having up to 6 ring members;
wherein the alkyl, alkenyl or alkynyl in R₁ or R_{1′}, if substituted, is substituted with one or more substituents selected from the group consisting of —OR₃, halogen, —CN, haloalkyl, haloalkoxy and —NR₃R_{3′″};
wherein
R₃ is selected from the group consisting of hydrogen, unsubstituted C₁₋₆ alkyl, unsubstituted C₂₋₆ alkenyl and unsubstituted C₂₋₆ alkynyl;
and wherein R_{3′″} is selected from the group consisting of hydrogen, unsubstituted C₁₋₆ alkyl, unsubstituted C₂₋₆ alkenyl, unsubstituted C₂₋₆ alkynyl and -Boc;
R₂ is selected from the group consisting of hydrogen, halogen, —R₄, —OR₄, —NO₂, —NR₄R_{4′″}, —NR₄C(O)R_{4′}, —NR₄S(O)₂R_{4′}, —S(O)₂NR₄R_{4′}, —NR₄C(O)NR_{4′}R_{4″}, —SR₄, —S(O)R₄, —S(O)₂R₄, —OS(O)₂R₄, —CN, haloalkyl, haloalkoxy, —C(O)OR₄, —C(O)NR₄R_{4′}, —OCH₂CH₂OH, —NR₄S(O)₂NR_{4′}R_{4″} and —C(CH₃)₂OR₄; wherein
R₄, R_{4′} and R_{4″} are independently selected from the group consisting of hydrogen, unsubstituted C₁₋₆ alkyl, unsubstituted C₂₋₆ alkenyl, and unsubstituted C₂₋₆ alkynyl;
R_{4′″} is selected from the group consisting of hydrogen, unsubstituted C₁₋₆ alkyl, unsubstituted C₂₋₆ alkenyl, unsubstituted C₂₋₆ alkynyl and -Boc.

17. The compound according to claim 16, wherein when X is —CH₂N(R_{1′})— or —C(O)N(R₁)—, R₁ and R_{1′} taken together with the connecting N atom may form a substituted or unsubstituted 6-membered heterocycyl.

18. The compound according to claim 15, wherein R₁ is selected from the group consisting of hydrogen, substituted or unsubstituted C₁₋₆ alkyl, substituted or unsubstituted cycloalkyl and substituted or unsubstituted heterocyclyl.

19. The compound according to claim 18, wherein R₁ is selected from the group consisting of hydrogen, substituted or unsubstituted methyl, substituted or unsubstituted ethyl, substituted or unsubstituted isopropyl, substituted or unsubstituted isobutyl, substituted or unsubstituted cyclopropyl and substituted or unsubstituted pyridine.

20. The compound according to claim 15, wherein R₂ is hydrogen or —OR₄.

21. The compound according to claim 20, wherein R₂ is —OH or —O-methyl.

22. The compound according to claim 21, wherein R₂ is in the meta position.

23. The compound according to claim 15, wherein the 6-membered heterocyclyl in R₁-R_{1″} is a substituted or unsubstituted group selected from piperidine, piperazine and morpholine.

24. The compound according to any one of claim 15, wherein when X is —CH₂N(R_{1′})— or —C(O)N(R_{1′})— and when R₁ and R_{1′} taken together with the connecting N—[CH₂]ₙ atoms or N atom form a substituted or unsubstituted 6-member heterocyclyl, —X—[CH₂]ₙ—R₁ or —X—R₁ is represented by 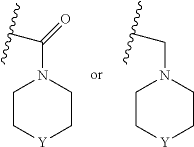
respectively,
with Y being selected from —O—, —N(R_{1″})— or —CH(R_{1″})— and
R_{1″} being selected from the group consisting of hydrogen, substituted or unsubstituted C₁₋₆ alkyl, substituted or unsubstituted C₂₋₆ alkenyl, or substituted or unsubstituted C₂₋₆ alkynyl, substituted or unsubstituted cycloalkyl, substituted or unsubstituted aryl, or substituted or unsubstituted heterocyclyl.

25. The compound according to claim 24, wherein R_{1″} is selected from the group consisting of hydrogen, unsubstituted C₁₋₄ alkyl, and unsubstituted aryl.

26. The compound according to claim 15, which is selected from the group consisting of:
3-ethyl-3-(3-methoxyphenyl)-1-(2-(4-methylpiperazin-1-yl)ethyl)azepane,
3-ethyl-3-(3-methoxyphenyl)-1-(2-(4-phenylpiperazin-1-yl)ethyl)azepane,
4-(2-(3-Ethyl-3-(3-methoxyphenyl)azepan-1-yl)ethyl)morpholine,
3-ethyl-3-(3-methoxyphenyl)-1-(2-(piperidin-1-yl)ethyl)azepane,
1-(2-ethoxyethyl)-3-ethyl-3-(3-methoxyphenyl)azepane,
3-(3-ethyl-1-(2-(4-methylpiperazin-1-yl)ethyl)azepan-3-yl)phenol,
3-(3-ethyl-1-(2-(4-phenylpiperazin-1-yl)ethyl)azepan-3-yl)phenol,
3-(3-ethyl-1-(2-morpholinoethyl)azepan-3-yl)phenol,
3-(3-ethyl-1-(2-(piperidin-1-yl)ethyl)azepan-3-yl)phenol,
3-(3-ethyl-1-(2-hydroxyethyl)azepan-3-yl)phenol,
3-(1-(2-ethoxyethyl)-3-ethylazepan-3-yl)phenol,
3-(3-ethyl-1-(2-isopropoxyethyl)azepan-3-yl)phenol,
2-(3-ethyl-3-(3-hydroxyphenyl)azepan-1-yl)-1-(piperidin-1-yl)ethanone,
3-(3-ethyl-1-(2-(pyridin-3-yloxy)ethyl)azepan-3-yl)phenol,
2-(3-ethyl-3-(3-hydroxyphenyl)azepan-1-yl)-N,N-dimethylacetamide,
2-(3-ethyl-3-(3-hydroxyphenyl)azepan-1-yl)-1-morpholinoethanone,
3-(3-ethyl-1-(2-methoxyethyl)azepan-3-yl)phenol,
3-(1-(2-cyclopropoxyethyl)-3-ethylazepan-3-yl)phenol,
3-(3-ethyl-1-(2-(2-hydroxy-2-methylpropoxy)ethyl)azepan-3-yl)phenol,
(S)-3-(1-(2-ethoxyethyl)-3-ethylazepan-3-yl)phenol,
(R)-3-(1-(2-ethoxyethyl)-3-ethylazepan-3-yl)phenol,
3-[1-(2-Ethoxyethyl)-3-ethylazepan-3-yl]aniline,
N-{3-[1-(2-Ethoxyethyl)-3-ethylazepan-3-yl}phenyl)methanesulfonamide,
N-{3-[1-(2-Ethoxyethyl)-3-ethylazepan-3-yl]phenyl}propane-2-sulfonamide,
optionally as a stereoisomer, including enantiomers and diastereomers, a racemate, or a mixture of at least two stereoisomers, including enantiomers and/or diastereomers, in any mixing ratio, or a corresponding salt thereof, or a corresponding solvate thereof.

27. A process for the preparation of the compound of Formula (I) according to claim 15, which process comprises the reduction of a compound of Formula XI 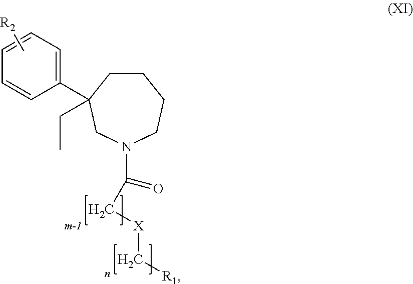
or the alkylation of a compound of Formula IX 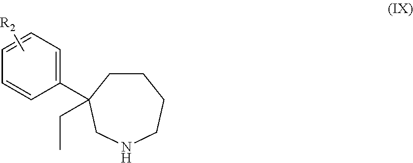
with a compound of formula Xa 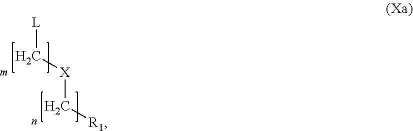
or the reductive amination of a compound of Formula IX 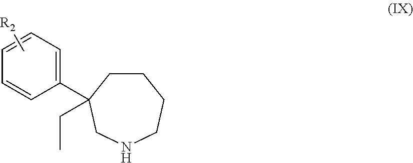
with a compound of formula Xb 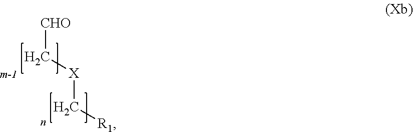
wherein R₁, R₂, X, m and n are as defined in claim 15 and L is a leaving group.

28. A process for the preparation of the compound of Formula (I′) according to claim 16, which process comprises the reduction of a compound of Formula XI′ 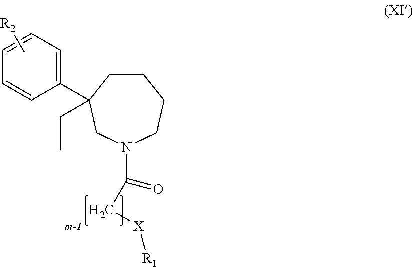
or the alkylation of a compound of Formula IX 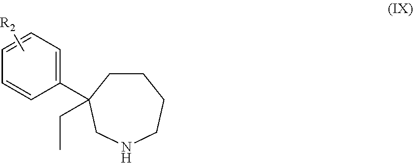
with a compound of formula Xa′ 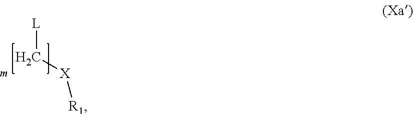
or the reductive amination of a compound of Formula IX 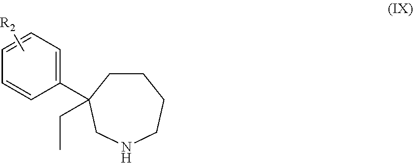
with a compound of formula Xb′ 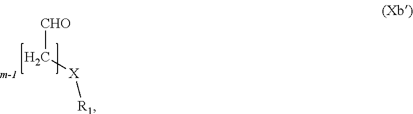
wherein R₁, R₂, X, m and n are as defined in claim 16 and L is a leaving group.

29. A process for the preparation of the compound of Formula (I) according to claim 15, employing a compound of formula II, III, IV, V, VI, VII, VIII, IX, Xa, Xa′, Xb, Xb′, Xc, Xc′, XI or XI′, 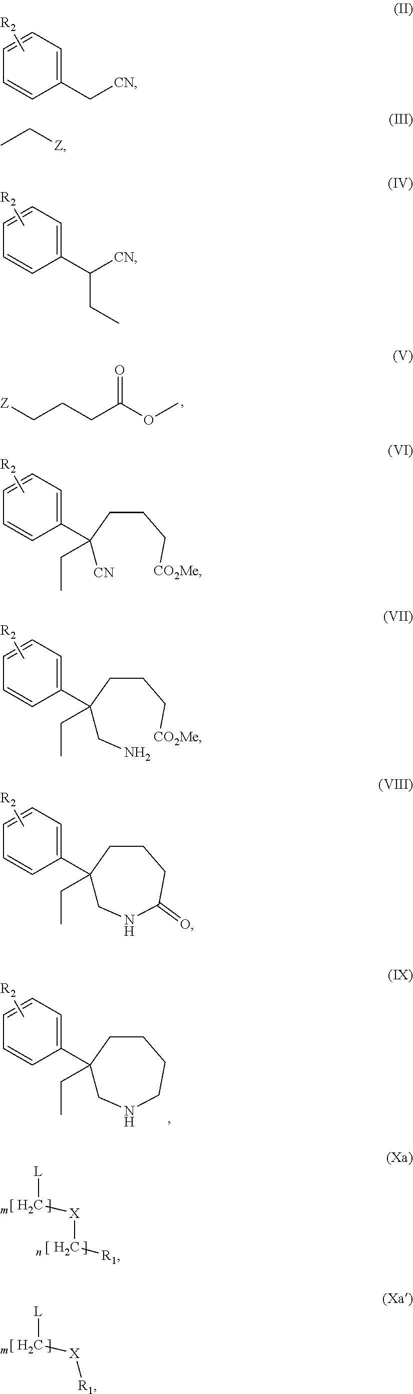 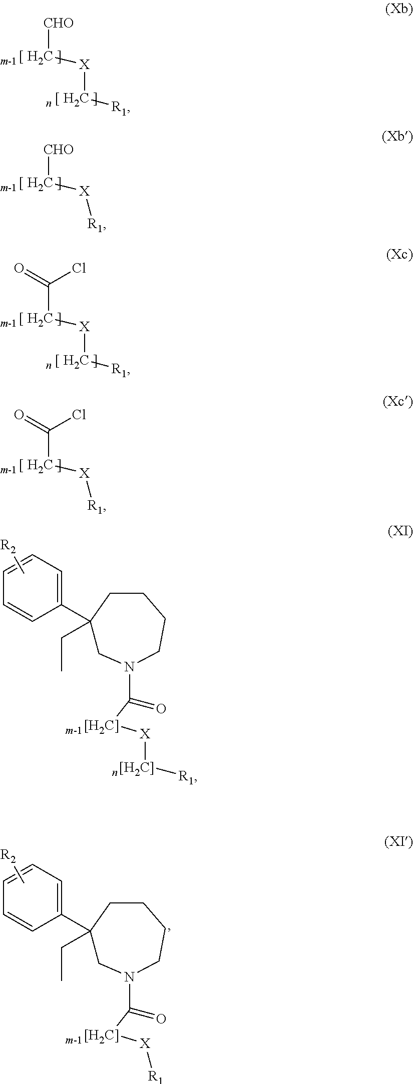
wherein R₁, R₂, X, m and n are as defined in claim 15 and Z is chloro, bromo or iodine.

30. A pharmaceutical composition which comprises the compound according to claim 15, or a pharmaceutically acceptable salt thereof, and a pharmaceutically acceptable carrier, adjuvant or vehicle.

31. A method of treating pain in a subject in need thereof, comprising administration of an effective amount of the compound according to claim 15.

32. The method according to claim 15, wherein the pain is selected from the group consisting of medium to severe pain, visceral pain, chronic pain, cancer pain, migraine, inflammatory pain, acute pain, neuropathic pain, allodynia and hyperalgesia.
